(19) United States
(12) Patent Application Publication
(10) Pub. No.: US 2006/0121607 A1
(43) Pub. Date: Jun. 8, 2006

(54) Compositions and methods for neural differentiation of embryonic stem cells

(76) Inventors: Thomas Schulz, Athens, GA (US); Steven L. Stice, Athens, GA (US); Brian G. Condie, Athens, GA (US); Bruce Davidson, Adelaide (AU)

Correspondence Address: SUTHERLAND ASBILL & BRENNAN LLP, 999 PEACHTREE STREET, N.E., ATLANTA, GA, 30309, US

(21) Appl. No.: 10/524,157

(22) PCT Filed: Aug. 8, 2003
(86) PCT No.: PCT/US03/24864

§ 371 Date: Aug. 22, 2005

(30) Foreign Application Priority Data
Aug. 8, 2002 (US) 60401968
Mar. 31, 2003 (US) 60459090
May 9, 2003 (AU) AU03/00552

(51) Int. Cl. C12N 5/08

(52) U.S. Cl. 435/368

(57) ABSTRACT

The present invention provides compositions and methods for human neural cell production. More particularly, the present invention provides cellular differentiation methods employing an essentially serum free MEDII conditioned medium for the generation of human neural cells from pluripotent and multipotent human stem cells.

### BACKGROUND OF THE INVENTION

1. Field of the Invention

This invention relates generally to human neural cells and to differentiated or partially differentiated cells derived therefrom. The present invention also relates to methods of producing, differentiating and culturing the cells of the invention, and to uses thereof.

2. Background Art

In the human and in other mammals, formation of the blastocyst, including development of inner cell mass (ICM) cells and their progression to pluripotent cells of the primitive ectoderm, and subsequent differentiation to form the embryonic germ layers and differentiated cells, follow a similar developmental process.

Embryonic stem (ES) cells represent a powerful model system for the investigation of mechanisms underlying pluripotent cell biology and differentiation within the early embryo, as well as providing opportunities for genetic manipulation of mammals and resultant commercial, medical and agricultural applications. Furthermore, appropriate proliferation and differentiation of ES cells can be used to generate an unlimited source of cells suited to transplantation for treatment of diseases that result from cell damage or dysfunction. Other pluripotent cells and cell lines including early primitive ectoderm-like (EPL) cells as described in International Patent Application WO 99/53021, in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer will share some or all of these properties and applications.

The successful isolation, long term clonal maintenance, genetic manipulation and germ-line transmission of pluripotent cells from species other than rodents has generally been difficult and the reasons for this are unknown. International Patent Application WO 97/32033 and U.S. Pat. No. 5,453,357 describe pluripotent cells including cells from species other than rodents. Human ES cells have been described in International Patent Application WO 96/23362, and in U.S. Pat. No. 5,843,780, and human EG cells have been described in International Patent Application WO 98/43679.

The ability to tightly control differentiation or form homogeneous populations of partially differentiated or terminally differentiated cells by differentiation in vitro of pluripotent cells has proved problematic. Current approaches involve the formation of embryoid bodies from pluripotent cells in a manner that is not controlled and does not result in homogeneous populations. Mixed cell populations such as those in embryoid bodies of this type are generally unlikely to be suitable for therapeutic or commercial use.

Uncontrolled differentiation produces mixtures of pluripotent stem cells and partially differentiated stem/progenitor cells corresponding to various cell lineages. When these ES-derived cell mixtures are grafted into a recipient tissue the contaminating pluripotent stem cells proliferate and differentiate to form tumors, while the partially differentiated stem and progenitor cells can further differentiate to form a mixture of inappropriate and undesired cell types. It is well known from studies in animal models that tumors originating from contaminating pluripotent cells can cause catastrophic tissue damage and death. In addition, pluripotent cells contaminating a cell transplant can generate various inappropriate stem cell, progenitor cell and differentiated cell types in the donor without forming a tumor. These contaminating cell types can lead to the formation of inappropriate tissues within a cell transplant. These outcomes cannot be tolerated for clinical applications in humans. Therefore, uncontrolled ES cell differentiation makes the clinical use of ES-derived cells in human cell therapies impossible.

Selection procedures have been used to obtain cell populations enriched in neural cells from embryoid bodies. These include manipulation of culture conditions to select for neural cells (Okabe et al., 1996 Dev. Biol. 176:300-312; and Tropepe et al., 2001 Neuron 30:65-78; O'Shea, 2002 Meth. in Mol. Biol. 198, 3-14), and genetic modification of ES cells to allow selection of neural cells by antibiotic resistance (Li et al., 1998 Current Biol. 8:971-974). Previously, one research group has demonstrated efficient differentiation of mouse and primate ES cells to TH⁺ neurons following co-culture with the PA6 stromal cell line, but this technique is not likely to be useful for cell therapy applications as it introduces xenograft issues associated with exposure to non-human cell lines and removal of potential PA6 cell contamination in subsequent cultures (Kawasaki et al., 2000 Neuron 28, 31-40; Kawasaki et al., 2002 Proc. Natl. Acad. Sci. USA, 99(3): 1580-1585). Furthermore, the PA6 differentiation procedure generated non-neural terminally differentiated cell types, such as retinal epithelial cells, reducing the usefulness of the cell cultures for cell therapy. In addition, McKay has demonstrated efficient differentiation of mouse ES cells to TH+ neurons, but this differentiation required over-expression of the Nurr-1 transcription factor in combination with exposure to Sonic Hedgehog and FGF8 (Kim et al., Nature 2002 418(6893):50-6). Furthermore, the McKay protocol involves a complex, five stage differentiation method for differentiation of mouse ES cells to neurons.

Another research group differentiated human ES cell derived embryoid bodies in 20% serum containing medium for 4 days followed by plating and selection/expansion of neural cell types in medium containing B27 and N2 supplements (serum free), EGF, FGF-2, PDGF-AA, and IGF-1 (Carpenter et al., 2001 Exper. Neuro. 172, 383-397). Carpenter et al. showed that neural progenitors could be enriched from this culture system by cell sorting or immunopanning using antibodies directed against polysialated NCAM or the cell surface molecule recognized by the A2B5 monoclonal antibody.

Another emerging approach to isolate neural cells is the use of transgenes that express a marker such as green fluorescent protein (GFP) under the control of a lineage specific promoter. The transgene allows cell sorting of the neural cells that differentiate from the ES cultures after retinoic acid and sonic hedgehog expression were used to induce the formation of spinal cord motor neurons (Steven Goldman, National Institutes of Health Symposium, NIH Research: Recent Progress and Future Promise of Human Embryonic Stem Cells, Jun. 12, 2003, abstract available at stemcells.nih.gov/news/symposiumSpeakers.asp#7 as of Jul. 30, 2003).

In all of these procedures, the differentiation of pluripotent cells in vitro does not involve biological molecules that direct differentiation in a controlled manner, that parallels or mimics the developmental stages of neural differentiation that occur in vivo. Similarly, in experiments examining neural differentiation from human ES cells, there is no way to control the neural differentiation, and the methods merely allow for the passive development of neural cell types (see Zhang et al., 2001 Nature Biotech 19(12): 1129-1133, and Reubinoff et al., 2001 Nature Biotech 19(12); 1134-40). Hence homogeneous, synchronous populations of neural cells with unrestricted neural differentiation capability are not produced, restricting the ability to derive essentially homogeneous populations of partially differentiated or differentiated neural cells.

Efficient neural differentiation of mouse embryonic stem cells in monolayer culture has recently been reported (Ying et al., 2003 Nature Biotechnology, 21:183-186). This previous study shows that adherent mouse ES cells can differentiate into neural cell types in a serum-free minimal medium. In contrast to the work described herein, the method described by Ying et al. produces neuronal cultures containing many GABAergic neurons and very few tyrosine hydroxylase expressing neurons. In addition the methods of Ying et al. are dependent on monolayer culture of the mouse ES cells.

Chemical inducers such as retinoic acid have also been used to form neural lineages from a variety of pluripotent cells including ES cells (Bain et al., 1995 Dev. Biol. 168:342-357; Strubing et al., 1995 Mech. Dev. 53, 275-287; Fraichard et al., 1995 J. Cell Sci. 108, 3181-3188; Schuldiner et al., 2001 Brain Res. 913, 201-205). However, the route of retinoic acid-induced neural differentiation has not been well characterized, and the repertoire of neural cell types produced appears to be generally restricted to ventral somatic motor, branchiomotor or visceromotor neurons (Renoncourt et al., 1998 Mech. Dev. 79:185-197).

Manually passaged HESC colonies are typically comprised of tightly packed, multilayered, undifferentiated HESCs, and variable levels of cells undergoing early differentiation. When present, these differentiating cells are observed on the edges of HESC colonies and are considered to be an indicator that the maintenance of the undifferentiated state of the colony is beginning to be compromised. This is undesirable as the presence of differentiating cells is likely to have a negative influence on maintaining the undifferentiated state of the remaining HESC, as the differentiating cells can produce factors that influence cellular differentiation. Furthermore, the presence of differentiated cells is likely to add randomness to differentiation procedures due to the stochastic presence of these cells and the differentiation signals or factors that they produce. Due to the three dimensional nature of the manually passaged HESC cultures, differentiating cells are also likely to be present in regions of the colonies where they cannot be detected or distinguished morphologically. As shown by Henderson et al. (Stem Cells, 2002, 20:329-337), SSEA3 or SSEA1 magnetic bead based sorting of cells confirms the likelihood of different cell populations within a culture akin to manually passaged HESC cultures. There is therefore a need to develop methods to passage HESCs that result in more uniform populations of undifferentiated or partially undifferentiated cells, and that are not based on morphological distinctions.

Previous publications report the transplantation of ES-derived neural cells into the ventricles of the fetal or newborn rat or mouse brain without the formation of tumors (Brustle et al., 1997 PNAS 94, 14809-14814, Zhang et al., 2001 Nature Biotech 19, 1129-1133). Although some of the cells in these studies do integrate into the host brain, many of the cells in the transplants form neural tube like structures within the lumen of the brain ventricle. Therefore, these previous studies do not lead to methods that can be readily applied to human cell therapy. Note that Reubinoff et al. (2001 Nature Biotech 19, 1134) also injected into the ventricles of newborn mice but did not report intraventricular masses of neural cells, omitting any mention of the presence or absence of such masses.

Neural stem cells and precursor cells have been derived from fetal brain and adult primary central nervous system tissue in a number of species, including rodent and human (e.g., see U.S. Pat. No. 5,753,506 (Johe), U.S. Pat. No. 5,766,948 (Gage), U.S. Pat. No. 5,589,376 (Anderson and Stemple), U.S. Pat. No. 5,851,832 (Weiss et al.), U.S. Pat. No. 5,958,767 (Snyder et al.) and U.S. Pat. No. 5,968,829 (Carpenter)). However, each of these disclosures fails to describe a predominantly homogeneous population of neural stem cells able to differentiate into all neural cell types of the central and peripheral nervous systems, and/or essentially homogeneous populations of partially differentiated or terminally differentiated neural cells derived from neural stem cells by controlled differentiation. Furthermore, it is not clear whether cells derived from primary fetal or adult tissue can be expanded sufficiently to meet potential cell and gene therapy demands. Neural stem cells derived from fetal or adult brain are established and expanded after the cells have committed to the neural lineage and in some cases after the cells have committed to neural sublineages. Therefore these cells do not provide the opportunity to manipulate the early differentiation processes that occur prior to neural commitment. Pluripotent stem cells provide access to these earliest stages of mammalian cellular differentiation opening additional options for cell expansion and directed development of the cells into desired lineages.

In summary, it has not been possible to control the differentiation of pluripotent cells in vitro, to provide homogeneous, synchronous populations of neural cells with unrestricted neural differentiation capacity. Similarly, methods have not been developed for the derivation of neural cells from pluripotent cells in a manner that parallels their formation during embryogenesis. In addition, current methods have relied upon the expression of foreign genes to drive neural differentiation of pluripotent stem cells (Kim et al., 2002 Nature 418:50-56). These limitations have restricted the ability to form essentially homogeneous, synchronous populations of partially differentiated and terminally differentiated neural cells in vitro, and have restricted their further development for therapeutic and commercial applications.

There is a need, therefore, to identify methods and compositions for the production of a population of cells enriched in neural stem cells and the products of their further differentiation, and in particular, human neural cells and their products.

### SUMMARY OF THE INVENTION

It is an object of the present invention to overcome, or at least alleviate, one or more of the difficulties or deficiencies associated with the prior art. In that regard, the present invention particularly provides a method of producing a human neural cell that includes the steps of: a) providing a pluripotent human cell; b) culturing the pluripotent human cell with an essentially serum free medium to form an embryoid body, and c) culturing the embryoid body with an essentially serum free cell differentiation environment until the human neural cell is produced. The present invention also provides a method of enriching a human cell culture for neural cells including the following steps: a) providing a pluripotent human cell culture; b) culturing the pluripotent human cell culture with an essentially serum free MEDII conditioned medium to form an embryoid body; and c) culturing the embryoid body with an essentially serum free cell differentiation environment until a human cell culture enriched in neural cells is produced. In certain preferred embodiments, the essentially serum free medium is a MEDII conditioned medium.

The MEDII conditioned medium described herein is preferably a Hep G2 conditioned medium that contains a bioactive component selected from the group consisting of: a large molecular weight extracellular matrix protein; a low molecular weight component comprising proline; a biologically active fragment of any of the aforementioned proteins or components; and an analog of any of the aforementioned proteins or components. The pluripotent human cell of the present invention can be selected from, but is not limited to, the group consisting of a human embryonic stem cell; a human ICM/epiblast cell; a human EPL cell; a human primitive ectoderm cell; a human primordial germ cell; and a human teratocarcinoma cell.

In other embodiments of the present invention, the methods described above further include the steps of dispersing the embryoid body to an essentially single cell suspension; culturing the essentially single cell suspension in a suspension culture and forming a second embryoid body by contacting the essentially single cell suspension with a second essentially serum free medium wherein the second essentially serum free medium is a MEDII conditioned medium; and contacting the second embryoid body with an essentially serum free cell differentiation environment until the human neural cell is produced, or the human cell culture enriched in neural cells is produced.

The invention provides a composition comprising a culture of neural cells, wherein the neural cells are preferably neural progenitor cells. The neural progenitor cells are characterized by the expression of nestin or vimentin, and their capacity to differentiate into cells of the neural lineage including neurons and glia. The neural cell types produced may include cells of the central or peripheral nervous system, including, but not limited to neurons, astrocytes, oligodendrocytes and Schwann cells. Neuron cell types produced in these cultures may express one or more neurotransmitter phentotypes. These include GABAergic neurons that express glutamate decarboxylase (GAD) or vesicular inhibitory amino acid transporter/vesicular gaba transporter (Viaat/Vgat); cholinergic neurons that express choline acetyltransferase (ChAT/CAT) or vesicular acetylcholine transporter (VAChT); glutamatergic neurons that express the vesicular glutamate transporter; glycinergic neurons that express the vesicular inhibitory amino acid transporter (Viaat/Vgat), noradrenergic neurons that express the norepinephrine transporter (NET); adrenergic neurons that express phenylmethanolamine N-methyl transferase (PNMT); serotonergic neurons that express tryptophan hydroxylase (TrH) or serotonin transporter (SERT); or histaminergic neurons that express histidine decarboxylase (HDC).

The invention further provides a composition comprising a culture of neural cells comprising neural cells derived in vitro from a pluripotent or multipotent cell. In preferred embodiments, these neural cells are capable of expressing one or more of the detectable markers for tyrosine hydroxylase (TH), vesicular monamine transporter (VMAT) dopamine transporter (DAT), and aromatic amino acid decarboxylase (AADC, also known as dopa decarboxylase). Preferably, the neural cell culture expresses all of the detectable markers for TH, VMAT, DAT, and AADC. Such a neural cell culture was not previously available and can be produced by the methods described herein or by other methods later developed.

The invention further provides methods of producing a partially differentiated pluripotent cell comprising culturing pluripotent cells on a layer of fresh feeder cells, wherein the fresh feeder cells are less than 2 days old, thereby inducing formation of a more differentiated pluripotent cell. In preferred embodiments, the fresh feeder cells are less than one day old, more preferably less than 12 hours old, or more preferably less than 6 hours old. In preferred embodiments, the more differentiated pluripotent cell is obtained from the central, or crater, region of the colony of pluripotent cells. In some embodiments, the more differentiated pluripotent cell expresses less Oct4 marker than an embryonic stem cell. The invention further provides a composition comprising these more differentiated pluripotent cells.

The invention further provides a method of treating a patient with a neural disease, comprising a step of administering to the patient a therapeutically effective amount of the neural cell or cell culture enriched in neural cells produced using the methods of the present invention.

The invention further provides for the human pluripotent cells, the human neural precursor cells and human neural cells produced using the methods of the invention.

### BRIEF DESCRIPTION OF THE DRAWINGS

FIGS. 1A and 1B show derived serum free embryoid bodies. Human embryonic stem cell colonies were dissected into uniform sized pieces and cultured in suspension. Solid serum free embryoid bodies (FIG. 1A) were distinguished from structured serum free embryoid bodies (FIG. 1B) after one week.

FIGS. 2A-2B show neural differentiation of serum free embryoid bodies. FIG. 2A shows proliferation of neural progenitor rosettes observed in serum free embryoid bodies seeded in the presence of 50% MEDII. FIG. 2B shows neurite network differentiation observed in serum free embryoid bodies seeded in the presence of 50% MEDII.

FIGS. 3A-3C show serum free embryoid bodies containing neural precursors. FIG. 3A shows phase contrast micrograph of seeded structured (left) and solid (right) serum free embryoid bodies. Greyscale images of fluorescent stainings with anti-Sox1 is shown in FIG. 3B and anti-Map2 antibodies is shown in FIG. 3C. Solid serum free embryoid bodies contain a high proportion of neural precursors (Sox1⁺ cells) and rare neurites (Map2⁺). Structured serum free embryoid bodies contain rosettes of semi-differentiated neural precursors (Sox1⁺/Map2⁺) and neurites (Map2⁺).

FIGS. 4A-4B show Map2 and nestin expression in rosettes. Radial Map2⁺ and nestin+ expression indicates a semi-differentiated progenitor/young neuron.

FIGS. 5A-5C show Sox1, Map2, and DAPI expression in serum free embryoid body differentiation. Map2 expression in FIG. 5A indicates semi-differentiated neurons (radial pattern) and differentiated neurons (network, dendritic stain), Sox1 expression in FIG. 5B indicates neural precursors, and DAPI in FIG. 5C is a DNA stain highlighting all nuclei in the field. Virtually all nuclei in the rosette are double stained for Sox1/DAPI, indicating a relatively pure population of neural progenitors (Sox1⁺) and/or semi-differentiated neural progenitors (Sox1⁺/Map2⁺).

FIGS. 6A-D show Sox1, nestin and Map2 expression in serum free embryoid body differentiation. A phase contrast micrograph of a region of a differentiating serum free embryoid body is shown in FIG. 6A. Rosette, neuron and other cell types are present. Greyscale images of fluorescent stainings with anti-nestin in FIG. 6B, anti-Sox1 in FIG. 6C and anti-Map2 in FIG. 6D are shown. Observed cell types are: neural progenitors (Nestin⁺/Sox1⁺) and/or semi-differentiated neurons (Nestin⁺/Sox1⁺/radial Map2⁺); differentiated neurons (Map2⁺), and other progenitor cell types (Nestin⁺).

FIG. 7 shows a network of anti-Tyrosine Hydroxylase stained neurons in a crater colony derived sfEBMs plated onto a polyornithine/laminin matrix

FIGS. 8A-8B show co-expression of Tyrosine Hydroxylase (FIG. 8A) and Map2 (FIG. 8B) shown by fluorescent immunostaining of neurons in a crater derived serum free embryoid body plated onto a polyornithine/laminin matrix.

FIGS. 9A-9B show crater derived serum free embryoid bodies at day 7 in suspension in (FIG. 9A) DMEM F12/N2/FGF-2 and (FIG. 9B) DMEM F12/N2/FGF-2/50% MEDII.

FIGS. 10A-D show expression of Oct4 protein in HESCs and serum free embryoid bodies. FIG. 10A shows high levels of Oct4 expression in a typical manually passaged HESC colony, with distinct nuclear expression in undifferentiated ES cells and no Oct4 in the unstained feeder layer surrounding the HESC colony. FIG. 10B shows a typical manually passaged HESC crater colony, showing high levels of Oct4 expression in the multilayered ring of undifferentiated cells surrounding the monolayer crater cells that express a low level of Oct4. Differentiating cells at the edge of the colony also express a low level of Oct4. FIG. 10C shows the expression of Oct4 in a seeded essentially serum free embryoid body, representative of what is seen when sfEBMs are derived from domed HESCs or monolayer crater cells. Regions of high level Oct4 expression persist and are indicative of residual nests of pluripotent cells maintained by local cell-cell signaling events. Neural rosettes in the same field are indicated as radially organized circles of nuclei by DAPI staining (FIG. 10D) and these neural precursor cells only express low levels of Oct4.

FIGS. 11A-L show immunostaining of SSEA4 selected trypsin passaged cells. FIGS. 11A and B show Oct4 and DAPI staining, respectively; FIGS. 11C and D show SSEA1 and DAPI staining, respectively; FIGS. 11E and F show SSEA3 and DAPI staining, respectively; Figs. G and H show SSEA4 and DAPI staining, respectively, FIGS. 11I and J show Tra-1-60 and DAPI staining, respectively; and FIGS. 11K and L show Tra-1-81 and DAPI staining respectively.

FIGS. 12A-D show Nestin expression in manually passaged and SSEA4 selected trypsin passaged cells. FIGS. 12A and B show Nestin and DAPI staining of manually passaged HESCs, respectively. The edge of a HESC colony is shown, showing that multilayered cells toward the center of the colony do not exhibit nestin expression (indicated by the dot in the lower right corner), while nestin expressing cells encircle the colony (indicated by the arrowhead), which are in turn surrounded by an outer ring of differentiating nestin+ cells (top left corner, indicated by the arrow). Figs. C and D show Nestin and DAPI staining of SSEA4 selected trypsin passaged HESCs, respectively. A substantially uniform distribution of nestin is exhibited.

FIGS. 13A-D show enhanced neural differentiation of SSEA4 selected trypsin passaged HESCs in response to MEDII. Serum free embryoid bodies were derived, exposed to 10 μM S18 from day 13 to day 17, seeded at day 18 and fixed for immunostaining at day 23. A and B show TH immunostaining and DAPI staining, respectively, of serum free embryoid bodies grown in FGF2. The proportion of TH⁺ cells and distribution of the network of the dopaminergic neural projections was considerably enhanced over what had previously been observed with serum free embryoid bodies derived from manually passaged HESCs. Up to 30-70% by area of the sfEBs contained TH⁺ neurons, as opposed to less than ˜20% for crater derived sfEBMs. Significant regions of the seeded embryoid bodies did not contain neurons. C and D show TH immunostaining and DAPI staining, respectively, of serum free embryoid bodies grown in FGF2/MEDII. A very high proportion of the culture, typically >90% of the area of a seeded sfEBM piece, consisted of TH⁺ neurons and the differentiation of these cells was enhanced, as they exhibited far more developed neural processes. Non-neural regions of the culture were significantly reduced. The proportion of neural rosettes appeared to be far greater in cultures exposed to MEDII.

FIGS. 14A-F show high efficiency dopaminergic differentiation. SSEA4 selected trypsin passaged HESCs were differentiated in response to MEDII to generate a very high proportion of TH⁺ neurons. Serum free embryoid bodies were derived, exposed to 10 μM S18 from day 13 to day 17, seeded at day 18 and fixed for immunostaining at day 23. A and B show βIII-Tubulin and DAPI staining, respectively, of a seeded sfEBM. The boxes mark the regions shown at increased magnification in C-F. C and E show an increased magnification of the TH immunostaining, and D and F show an increased magnification of the βIII-Tubulin immunostaining. A very high proportion, typically 90% or greater of the neurons express TH.

FIGS. 15A-B show a comparison of TH⁺ and Hoffman optics images of neural extensions in a region of a serum free embryoid body grown in 4 ng/ml FGF2. Serum free embryoid bodies were derived, exposed to 10 μM S18 from day 13 to day 17, seeded at day 18 and fixed for immunostaining at day 23. A very high proportion of neurons express TH.

FIGS. 16A-D show expression of TH and VMAT in sfEBM cultures. sfEBMs were derived, exposed to 10 μM S18 from day 13 to day 17, seeded at day 18 and fixed for immunostaining at day 23. A and C show VMAT expression at 40× and 20× magnification respectively. B and D show TH expression at 40× and 20× magnification respectively. TH⁺/VMAT⁻, TH⁻/VMAT⁺ and TH⁺/VMAT⁺ cells could be observed.

FIGS. 17A-B illustrate the dopamine release assay. FIG. 17A is a schematic representation of the purification, modification and competitive enzyme linked immunoassay. Dopamine (D) is released from cultured neurons by depolarization with KCl, D is then is purified with a cis-diol affinity resin and acylated to N-acyldopamine (D^{a}). D^{a} remains in suspension and is modified to N-acyl-3-Methoxytyamine (m), which competes with solid phase D for a limited number of anti-dopamine antibody binding sites. Free antigen and antibody are removed by washing, and antibody bound to solid phase D is detected with a secondary antibody-peroxidase conjugate. There is an inverse correlation between the amount of D in the samples and detected signal. The amount of D in the sample is established from a standard curve. FIG. 17B shows a determination of dopamine released from sfEBM samples, which had been derived, seeded to polyornithine/laminin coated slides, at day 25 and cultured to day 30 prior to depolarization. The cultures released approximately 2650 pg/ml of dopamine into the depolarizing medium (dot and vertical line). This value was within the range of the standard curve (dots representing 0, 150, 600, 2400, 9600, 38400 pg/ml dopamine) and fell between two unknown control samples from the kit (arrows).

FIGS. 18A-E show the neural differentiation of SSEA4 selected bulk passaged cells cultured as serum free embryoid bodies in FGF2 and Proline. sfEBP were derived and cultured for 10 or 17 days, and seeded to polyornithine/laminin for 5 days. FIGS. 18A, and B show seeded sfEBPs at day 15 stained with DAPI and anti-βIII-Tubulin, respectively, at 10× magnification. FIGS. 18C, D, and E show seeded sfEBPs at day 22 stained with DAPI, anti-βIII-Tubulin and anti-TH, respectively, at 40× magnification.

FIG. 19 shows neural differentiation of SSEA4 selected bulk passaged cells cultured as serum free embryoid bodies in minimal medium without FGF2, MEDII or L-Proline. Serum free embryoid bodies were seeded at day 21, fixed at day 25 and immunostained with anti-βIII-Tubulin and imaged at 10× magnification.

FIGS. 20A-B show, whole mount immunostaining and confocal analysis of 50 μM L-Proline sfEBP at day 27 after derivation. Different sfEBPs are shown in these images. FIG. 20A shows anti-βIII-Tubulin immunostaining, detected with an Alexa 488 labeled secondary antibody and 1 μm confocal section at 40× magnification. Complex networks of βIII-Tubulin positive neuronal extensions were detected. A non-staining neural rosette is indicated by the asterisk, and βIII-Tubulin positive cell bodies are indicated by arrowheads. FIG. 20B shows a DAPI stained sfEBP imaged at 1 μm sections by a 2-Photon laser confocal at 40× magnification. A large proportion of the sfEBP consists of the elongated, closely packed, radial, neural rosette nuclei. The two dashed ovals surround a rosette and indicate its central proliferative core, where mitotic figures are localized within rosettes.

### DETAILED DESCRIPTION OF THE INVENTION

Applicant has demonstrated that contacting pluripotent human cells such as human ES cells, with an essentially serum free medium results in the formation of a human neural cell type with greater efficiency or frequency. The invention provides a neural cell culture composition characterized by a variety of properties, heretofore unavailable. The invention further provides a partially differentiated pluripotent cell by culturing pluripotent cells on a fresh feeder cell layer, and methods of obtaining the same.

The present invention particularly provides a method of producing a human neural cell that includes the steps of: a) providing a pluripotent or multipotent human cell; and b) contacting the pluripotent or multipotent human cell with an essentially serum free cell differentiation environment until the human neural cell is produced. The present invention additionally provides a method of enriching a human cell culture for neural cells comprising the steps of: a) providing a pluripotent or multipotent human cell culture; and b) contacting the pluripotent or multipotent human cell culture with an essentially serum free cell differentiation environment until a human cell culture enriched in neural cells is produced.

In preferred embodiments of the above methods, an embryoid body is formed upon culturing the pluripotent or multipotent human cell or cell culture with an essentially serum free medium, and as a subsequent step, the cells from the embryoid body are contacted with an essentially serum free cell differentiation environment until a human neural cell or human cell culture enriched in neural cells is produced. In preferred embodiments, the essentially serum free medium is a MEDII conditioned medium as defined herein. In other embodiments, the embryoid body is contacted with one or more differentiation medium or cell differentiation environments after being removed from the essentially serum free cell differentiation environment until a human neural cell or human cell culture enriched in neural cells is produced, wherein each medium or environment is appropriate to the cell types as they appear from the preceding cell type. It is to be understood that the absence of the term “differentiation” when describing a MEDII conditioned medium does not indicate that the MEDII conditioned medium can not also be considered a “differentiation” medium. In certain embodiments, the essentially serum free medium preferably is also essentially LIF free.

In another preferred embodiment of the above method, an embryoid body is formed upon culturing the pluripotent or multipotent human cell or cell culture with an essentially serum free MEDII conditioned medium, the cells from the embryoid body are contacted with an essentially serum free cell differentiation environment, the embryoid body is dispersed to an essentially single cell suspension, the single cell suspension is cultured in suspension culture, a second embryoid body is formed by contacting the essentially single, cell suspension with a second essentially serum free medium where the second essentially serum free medium is a MEDII conditioned medium; and the second embryoid body is, contacted with an essentially serum free cell differentiation environment until the human neural cell is produced or human cell culture enriched in neural cells is produced. The second essentially serum free media can comprise DMEM/F12, FGF (e.g., FGF-2) and MEDII conditioned media. In a preferred embodiment the MEDII conditioned media comprises 10-75% of the second essentially serum free media. More preferably, the MEDII conditioned media comprises 40-60% of the second essentially serum free media, and most preferably the MEDII conditioned media comprises 50% of the second essentially serum free media. In a preferred embodiment the MEDII conditioned media comprises approximately 1-30 ng/ml of FGF-2, more preferably approximately 2-10 ng-ml of FGF-2, and most preferably approximately 4 ng/ml of FGF-2.

Applicant has demonstrated that culturing human cell populations comprising pluripotent human cells by selecting the cells with an antibody directed to a pluripotent cell marker, and/or passaging the cells with a protease treatment results in the formation of a human pluripotent cell type that expresses cell markers characteristic of human embryonic stem cells, and also expresses nestin in a substantially uniform manner. When these cells are cultured with MEDII, they form neural cells with greater homogeneity than observed in a pluripotent human cell population that is not cultured with MEDII. When these cells are cultured with a minimal medium that optionally comprises proline, they form neural cells with greater homogeneity than observed in a pluripotent human cell population that is not cultured with minimal medium. This differentiation protocol has the capacity to be performed on a large scale, free of exposure to non-human cell types, to generate a high proportion of dopaminergic neurons, in the absence of residual pluripotent cells.

The invention provides a composition comprising a culture of neural cells, wherein the neural cells are preferably neural progenitor cells. The neural progenitor cells are characterized by the expression of nestin or vimentin, and their capacity to differentiate into cells of the neural lineage including neurons and glia. The neural cell types produced may include cells of the central or peripheral nervous system, including, but not limited to neurons, astrocytes, oligodendrocytes and Schwann cells. Neuron cell types produced in these cultures may express one or more neurotransmitter phentotypes. These include GABAergic neurons that express glutamate decarboxylase (GAD) or vesicular inhibitory amino acid transporter/vesicular gaba transporter (Viaat/Vgat); cholinergic neurons that express choline acetyltransferase (ChAT/CAT) or vesicular acetylcholine transporter (VAChT); glutamatergic neurons that express the vesicular glutamate transporter; glycinergic neurons that express the vesicular inhibitory amino acid transporter (Viaat/Vgat), noradrenergic neurons that express the norepinephrine transporter (NET); adrenergic neurons that express phenylmethanolamine N-methyl transferase (PNMT); serotonergic neurons that express tryptophan hydroxylase (TrH) or serotonin transporter (SERT); or histaminergic neurons that express histidine decarboxylase (HDC).

In one embodiment, the neural cell produced by culturing the differentiated pluripotent human cell is therapeutically transplanted into the brain of a subject. The cell culture of the present invention form teratomas at a greatly reduced frequency than if the culture was not treated with a serum free differentiation environment and/or passaged using a protease treatment. In a preferred embodiment, the cell culture of the present invention does not induce the formation of teratomas at a significant rate.

The present invention further provides a method of culturing a human pluripotent cell, comprising the steps: a) selecting a human pluripotent cell using an anti-SSEA4 antibody; and b) maintaining a culture of the cell by passaging the cell using a protease treatment, wherein the cells of the culture express SSEA3, SSEA4, Oct4, Tra-1-60, Tra-1-80, and express nestin substantially uniformly. As used herein, the term “substantially uniformly” refers to the expression pattern of a cellular marker when a colony of cells is examined for expression of that marker. If there is “substantially uniform” expression of a marker, generally most of the cells of the colony express the marker. For example, if the center of an HESC colony does not express a marker, but the marker is expressed in most of the cells in the remainder of the colony, the marker is not expressed in a substantially uniform manner. Preferably, greater than 90% of the cells of a colony express the marker, more preferably, greater than 91%, 92%, 93%, 94%, 95%, 96%, 97%, 98%, of the cells of the colony express the marker, and still more preferably, greater than 99% of the cells of the colony express the marker.

In a preferred embodiment, the protease treatment comprises the sequential use of Collagenase and trypsin. In one embodiment, Collagenase is used at a concentration of from approximately 0.1 mg/ml to approximately 10 mg/ml, more preferably from a concentration of from approximately 0.5 mg/ml to approximately 5 mg/ml, and most preferably at a concentration of from approximately 1 mg/ml to 2 mg/ml. The invention contemplates that Collagenase may be used for approximately 1 minute to 10 minutes, more preferably from approximately 2 minutes to 8 minutes, and most preferably for approximately 4 minutes to 6 minutes.

In another embodiment, trypsin is used at a concentration of from approximately 0.001% to 1%, more preferably at a concentration of from approximately 0.01% to 0.1%, and most preferably at a concentration of approximately 0.05%. The invention contemplates that trypsin may be used for approximately 1 second to 5 minutes, more preferably for approximately 5 seconds to 2 minutes, more preferably for approximately 10 seconds to 1 minute, and most preferably for approximately 30 seconds.

In a further preferred embodiment, Collagenase is used at a concentration of approximately 1 mg/ml for approximately 5 minutes, and trypsin is used at a concentration of approximately 0.05% for approximately 30 seconds.

The methods of the present invention further encompass providing a human cell culture enriched in neural cells, comprising the formation of an embryoid body that comprises a human pluripotent cell culture that expresses SSEA3, SSEA4, Oct4, Tra-1-60, Tra-1-80, and expresses nestin substantially uniformly. In one embodiment, the human pluripotent cell culture is provided using a protease passaging treatment. In another embodiment, the human pluripotent cell culture is provided using antibody selection and protease passaging treatment. In another embodiment, the human pluripotent cell culture is provided using antibody selection. In preferred embodiments of the invention, the antibody selection is performed using an anti-SSEA4 antibody. In a further preferred embodiment, the protease passaging treatment comprises the use of Collagenase at a concentration of approximately 1 mg/ml for approximately 5 minutes, and the subsequent use of trypsin at a concentration of approximately 0.05% for approximately 30 seconds.

In a further preferred embodiment, the method of providing a human cell culture enriched in neural cells comprises the formation of an embryoid body by culturing a human pluripotent cell culture with an essentially serum free medium. In a preferred embodiment, the essentially serum free medium is a MEDII conditioned medium as defined herein. In another preferred embodiment, the essentially serum free medium is a minimal medium that optionally comprises proline.

In other embodiments of the present invention, it is not required that an embryoid body is formed upon contacting the pluripotent or multipotent human cell or cell culture with an essentially serum free MEDII conditioned medium. In these embodiments, a pluripotent or multipotent human cell or cell culture is contacted with a MEDII conditioned medium, and as a subsequent step, the resultant cells can be contacted with an essentially serum free cell differentiation environment until a human neural cell or human cell culture enriched in neural cells is produced. In other embodiments, the resultant cells are subsequently contacted with one or more differentiation medium after being removed from the essentially serum free cell differentiation environment until a human neural cell or human cell culture enriched in neural cells is produced, wherein each medium is appropriate to the cell types as they appear from the preceding cell type. In other embodiments the resultant cells are subsequently contacted with a different cell differentiation environment after being removed from the essentially serum free cell differentiation environment until a human neural cell or human cell culture enriched in neural cells is produced, wherein each cell differentiation environment is appropriate to the cell types as they appear from the preceding cell types.

The invention further provides a composition comprising a culture of neural cells comprising a neural cell derived in vitro from a pluripotent or multipotent cell. In preferred embodiments, the neural cells are human cells. In preferred embodiments, the neural cell is capable of expressing one or more of the detectable markers for tyrosine hydroxylase (TH), vesicular monamine transporter (VMAT) dopamine transporter (DAT), and aromatic amino acid decarboxylase (AADC, also known as dopa decarboxylase). Preferably, the cultured cell expresses detectable markers for TH, VMAT, DAT, and AADC. In other embodiments, the neural cell is capable of expressing one or more of the detectable markers for nestin, Sox1, and Map2. Preferably, the cultured cell expresses detectable markers for nestin, Sox1, and Map2. Such a culture of cells not previously provided in the art can be produced by the methods described herein or by other methods later developed.

The present invention further provides a method of culturing a human pluripotent cell, comprising the steps: a) selecting a human pluripotent cell using an anti-SSEA4 antibody; and b) maintaining a culture of the cell by passaging the cell using a protease treatment, wherein the cells of the culture express SSEA3, SSEA4, Oct4, Tra-1-60, Tra-1-80, and express nestin substantially uniformly. As used herein, the term “substantially uniformly” refers to the expression pattern of a cellular marker when a colony of cells is examined for expression of that marker. If there is “substantially uniform” expression of a marker, generally most of the cells of the colony express the marker. For example, if the center of an HESC colony does not express a marker, but the marker is expressed in most of the cells in the remainder of the colony, the marker is not expressed in a substantially uniform manner. Preferably, greater than 90% of the cells of a colony express the marker, more preferably, greater than 91%, 92%, 93%, 94%, 95%, 96%, 97%, 98%, of the cells of the colony express the marker, and still more preferably, greater than 99% of the cells of the colony express the marker.

It is to be understood that as used in the specification and in the claims, “a” or “an” can mean one or more, depending upon the context in which it is used. Thus, for example, reference to “a cell” can mean that at least one cell can be utilized. As also used herein, the term “MEDII conditioned medium” refers to a medium comprising one or more bioactive components as described herein. In a preferred embodiment, the bioactive component is derived from a hepatic or hepatoma cell or cell line culture supernatant. The hepatic or hepatoma cell or cell line can be from any species, however, preferred cell lines are mammalian or avian in origin. The hepatic or hepatoma cell line can be selected from, but is not limited to, the group consisting of: a human hepatocellular carcinoma cell line such as a Hep G2 cell line (ATCC HB-8065) or Hepa-1c1c-7 cells (ATCC CRL-2026); a primary embryonic mouse liver cell line; a primary adult mouse liver cell line; a primary chicken liver cell line; and an extraembryonic endodermal cell line such as END-2 and PYS-2. A particularly preferred cell line is the Hep G2 cell line (ATCC HB-8065). A description of the isolation of an essentially serum free MEDII medium from a Hep G2 cell line is provided in Example 1 below. In one embodiment of the present invention, the MEDII medium is derived from a Hep G2 cell line and contains supplements of FGF2 and hLIF.

As used herein, the terms “bioactive component” and “bioactive factor” refer to any compound or molecule that induces a pluripotent cell to follow a differentiation pathway toward an EPL cell or a neural cell. Alternatively, the bioactive component may act as a mitogen or as a stabilizing or survival factor for a cell differentiating towards an EPL cell or neural cell. A bioactive component from the conditioned medium may be used in place of the MEDII conditioned medium in any embodiment described herein. While the bioactive component may be as described below, the term is not limited thereto. The term “bioactive component” as used herein includes within its scope a natural or synthetic molecule or molecules which exhibit(s) similar biological activity, e.g. a molecule or molecules which compete with molecules within the conditioned medium that bind to a receptor on ES or EPL cells or their differentiation products in adherent culture, in embryoid bodies, or in non-adherent cultures, responsible for EPL or neural induction.

The MEDII conditioned medium described herein can comprise one or more bioactive components selected from the group consisting of a large molecular weight extracellular matrix protein; a low molecular weight component comprising proline; a biologically active fragment of any of the aforementioned proteins or components; and an analog of any of the aforementioned proteins or components. The large molecular weight extracellular matrix protein preferably has a molecular weight of greater than approximately, 10 kD, more preferably between approximately 100-500 kD and most preferably between approximately 210-250 kD as measured on a 10% reducing/denaturing polyacrylamide gel. In a further preferred embodiment, the large molecular weight extracellular matrix protein comprises a cellular fibronectin protein or a laminin protein. In one preferred embodiment, the bioactive component of the MEDII conditioned medium can be replaced, at least in part, by proline. Preferably proline is present in the cell culture medium at a concentration of from approximately 1 μM to approximately 1 M, more preferably from a concentration of from approximately 5 μM to approximately 500 μM, more preferably from approximately 10 μM to approximately 200 μM, and more preferably from approximately 25 μM to approximately 100 μM. In a preferred embodiment, proline is present in the cell culture medium at a concentration of approximately 50 μM. In addition, the MEDII conditioned medium may contain a neural inducing factor.

The low molecular weight component of the MEDII conditioned medium can comprise one or more proline residues or a polypeptide containing proline residues. As used herein, the term “polypeptide” refers to any of various amides that are derived from two or more amino acids by combination of the amino group of one acid with the carboxyl group of another and usually obtained by partial hydrolysis of proteins. In a preferred embodiment, the low molecular weight component is L-proline or a polypeptide including L-proline. The proline containing polypeptide preferably has a molecular weight of less than approximately 5 kD, more preferably less than approximately 3 kD. In a further preferred embodiment, the low molecular weight component is a polypeptide of between approximately 2-11 amino acids, more preferably of between approximately 2-7 amino acids and most preferably approximately 4 amino acids. The proline containing polypeptide can be selected from, but is not limited to, the following polypeptides: Pro-Ala, Ala-Pro, Ala-Pro-Gly, Pro-OH-Pro, Pro-Gly, Gly-Pro, Gly-Pro-Ma, Gly-Pro-Ma, Gly-Pro-OH-Pro, Gly-Pro-Arg-Pro (SEQ ID NO:1), Gly-Pro-Gly-Gly (SEQ ID NO:2), Val-Ala-Pro-Gly (SEQ ID NO:3), Arg-Pro-Lys-Pro (SEQ BD NO:4), and Arg-Pro-Lys-Pro-Gln-Gln-Phe-Phe-Gly-Leu-MetOH (SEQ ID NO:5).

In a preferred embodiment, the pluripotent or multipotent cell is a human cell. As used herein, the term “pluripotent human cell” encompasses pluripotent cells obtained from human embryos, fetuses or adult tissues. In one preferred embodiment, the pluripotent human cell is a human pluripotent embryonic stem cell. In another embodiment the pluripotent human cell is a human pluripotent fetal stem cell, such as a primordial germ cell. In another embodiment the pluripotent human cell is a human pluripotent adult stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. The term “multipotent” refers to a cell that is not terminally differentiated. The pluripotent human cell can be selected from the group consisting of a human embryonic stem (ES) cell; a human inner cell mass (ICM)/epiblast cell; a human primitive ectoderm cell, such as an early primitive ectoderm cell (EPL); a human primordial germ (EG) cell; and a human teratocarcinoma (EC) cell. The human pluripotent cells of the present invention can be derived using any method known to those of skill in the art. For example, the human pluripotent cells can be produced using dedifferentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021. Furthermore, the human pluripotent cells can be passaged using any method known of those to skill in the art, including, manual passaging methods, and bulk passaging methods such as antibody selection and protease passaging.

As used herein, the term “neural cell” includes, but is not limited to, a neurectoderm cell; an EPL derived cell; a glial cell; a neural cell of the central nervous system such as a dopaminergic cell, an astrocyte or an oligodendrocyte; a neural cell of the peripheral nervous system. As used herein, the term “neurectoderm” refers to undifferentiated neural progenitor cells substantially equivalent to cell populations comprising the neural plate and/or neural tube; or a partially differentiated neural progenitor cell. Neurectoderm cells are multipotent. Therefore, the use of the term “neural cell” in the context of the present invention means that the cell is at least more differentiated towards a neural cell type than the pluripotent cell from which it is derived. Also as used herein, producing a neural cell encompasses the production of a cell culture that is enriched for neural cells. In preferred embodiments, the term “enriched” refers to a cell culture that contains more than approximately 50%, 55%, 60%, 65%, 70%, 75%, 80%, 85%, 90%, or 95% of the desired cell lineage. In some embodiments of the present invention, neural cells express one or more of the detectable markers TH, VMAT, DAT, and AADC.

The step of contacting the human pluripotent cell or cell culture with the MEDII conditioned medium to produce embryoid bodies (EBs) or EPL cells can be conducted in any suitable manner. For example, EPL cells may be generated in adherent culture or as cell aggregates in suspension culture. EBs may be generated in suspension culture using the hanging drop technique or by culturing the cells on agarose coated plates. It is also to be understood that the step of contacting the embryoid body with an essentially serum free medium and/or an essentially serum free cell differentiation environment can also be conducted in any manner known to those of skill in the art. If the embryoid body is contacted with a further differentiation medium in addition to the essentially serum free differentiation medium, it is preferable that the essentially serum free medium is first removed.

As used herein “essentially serum free” refers to a medium that does not contain serum or serum replacement, or that contains essentially no serum or serum replacement. As used herein, “essentially” means that a de minimus or reduced amount of a component, such as serum, may be present that does not eliminate the improved bioactive neural cell culturing capacity of the medium or environment. For example, essentially serum free medium or environment can contain less than approximately 10%, 9%, 8%, 7%, 6%, 5%, 4%, 3%, 2%, or 1% serum wherein the presently improved bioactive neural cell culturing capacity of the medium or environment is still observed. In preferred embodiments of the present invention, the essentially serum free medium does not contain serum or serum replacement.

In one preferred embodiment, the essentially serum free differentiation medium comprises MEDII conditioned medium. Preferably, the essentially serum free differentiation medium comprises approximately 20% to approximately 80% MEDII conditioned medium, more preferably the essentially serum free differentiation medium comprises approximately 30% to approximately 70% MEDII conditioned medium, still more preferably the essentially serum free differentiation medium comprises approximately 40% to approximately 60% MEDII conditioned medium, and most preferably, the essentially serum free differentiation medium comprises approximately 50% MEDII conditioned medium. In other embodiments, the essentially serum free differentiation medium comprises approximately 20%, 30%, 35%, 40%, 45%, 50%, 55%, 60%, 65%, 70%, 75% or 80% MEDII conditioned medium.

As used herein “essentially LIF free” refers to a medium that does not contain leukemia inhibitory factor (LIF), or that contains essentially no LIF. “Essentially’ means that a de minimus or reduced amount of a component, such as LIF, may be present that does not eliminate the improved bioactive neural cell culturing capacity of the medium or environment. For example, essentially LIF free medium or environment can contain less than approximately 100, 75, 50, 40, 30, 10, 5, 4, 3, 2, or 1 ng/ml LIF, wherein the presently improved bioactive neural cell culturing capacity of the medium or environment is still observed. As used herein, the term “LIF” refers to leukemia inhibitory factor that is obtained or derived from any species, and is therefore not limited to human LIF.

In certain embodiments of the above methods, the MEDII conditioned medium is essentially free from LIF and from FGF2. In other embodiments of the above methods, the first or subsequent differentiation media are essentially free from LIF and from FGF2.

In one embodiment of the present inventions, the pluripotent cell or cell culture is cultured with a minimal medium. As used herein, the term “minimal medium” refers to a tissue culture medium that is preferably essentially free from FGF, proline, and/or MEDII. As used herein, “essentially free from FGF” or “essentially FGF free” refers to a tissue culture medium that contains less than approximately 10, 9, 8, 7, 6, 5, 4, 3, 2, 1, 0.5, 0.1, or 0.01 ng/ml of an FGF, wherein the presently improved bioactive neural cell culturing capacity of the medium or environment is still observed. Preferably, the minimal medium comprises less than 1 ng/ml of an FGF. As used herein, “essentially free from proline” or “essentially proline free” refers to a tissue culture medium that contains less than approximately 500 μM, 400 μM, 300 μM, 200 μM, 100 μM, 50 μM, 10 μM, 5 μM, or 1 μM of proline. In one embodiment, the minimal medium comprises less than 10 μM proline. In another embodiment, the minimal medium is supplemented with proline. When the minimal medium is supplemented with proline, preferably the proline is present at a concentration of less than 500 μM, 400 μM, 300 μM, 200 μM, 100 μM, 50 μM, 10 μM, 5 μM, or 1 μM of proline. In one embodiment, the minimal medium comprises approximately 50 μM proline. As used herein, “essentially free from MEDII” or “essentially MEDII free” refers to a tissue culture medium that contains less than approximately 50%, 40%, 30%, 20%, 10%, 5%, 4%, 3%, 2%, or 1% of MEDII, as defined herein. Preferably, a tissue culture medium essentially free from MEDII comprises less than 5% MEDII.

As used herein, an “essentially single cell culture” is a cell culture wherein during passaging, the cells desired to be grown are dissociated from one another, such that the majority of the cells are single cells, or two cells that remain associated (doublets). Preferably, greater than 50%, 60%, 70%, 80%, 90%, 95%, 96%, 97%, 98%, 99% or more of the cells desired to be cultured are singlets or doublets.

In a preferred embodiment of the above methods, a “feeder cell” is a cell that is co-cultured with a human pluripotent cell and maintains the human pluripotent cell in an undifferentiated or partially differentiated state. In a preferred embodiment of the above method, the conditioned medium is obtained from a feeder cell that maintains the human pluripotent cell in an undifferentiated or partially differentiated state. In one embodiment, the feeder cell is a mouse cell, such as a mouse embryonic fibroblast. In a preferred embodiment, the mouse embryonic fibroblast is mitotically inactivated, using methods well known to those of skill in the art. In another embodiment, the feeder cell is a human feeder cell. In certain embodiments, the human feeder cell is selected from the group consisting of a human fibroblast cell, a MRC-5 cell, a human embryonic kidney cell, a mesenchymal cell, an osteosarcoma cell, a keratinocyte, a chondrocyte, a Fallopian ductal epithelial cell, a liver cell, a cardiac cell, a bone marrow stromal cell, a granulosa cell, a skeletal muscle cell, and an aortic endothelial cell. In a more preferred embodiment the human feeder cell is selected from the group consisting of a skin keloid fibroblast cell, a fetal skin fibroblast cell, a bone marrow stromal cell, or a skeletal muscle cell.

The present invention contemplates that the feeder cell is a freshly plated feeder cell. As used herein, the term “freshly plated” means that the feeder cell has been allowed to attach to the tissue culture dish for less than 2 days. Preferably, the feeder cell has been plated for less than 18 hours, more preferably the feeder cell has been plated for less than 10 hours, more preferably the feeder cell has been plated for less than 6 hours, and most preferably, the feeder cell has been plated for less than 2 hours. In another embodiment, preferably the feeder cell has been plated for approximately 6 to 18 hours. In a preferred embodiment, HESC cultures that have been protease passaged and/or antibody selected are prepared for differentiation by seeding the cells at a defined density on feeder layers that are between approximately 6 to 18 hours old. In another embodiment, manually passaged HESC cultures are prepared for differentiation by seeding the cells at a defined density on feeder layers that are freshly plated. Seeding manually passaged HESCs on fresh feeder layers appears to cause a differentiation event that enables uniform neural rosette differentiation in suspension, and although morphological changes are not apparent, may also have a positive influence on the neural and DA differentiation of bulk passaged HESC.

As stated above, the present invention provides a method of producing a neural cell or enriching a culture for neural cells comprising the steps of: 1) providing a pluripotent human cell; 2) culturing the pluripotent human cell with an essentially serum free medium comprising MEDII conditioned medium and forming an embryoid body; and 3) culturing the embryoid body with an essentially serum free cell differentiation environment, and optionally contacting the embryoid body with one or more subsequent cell differentiation environments, until the neural cell or cell culture enriched in neural cells is produced.

It is to be understood that the step of contacting the pluripotent cell with the MEDII conditioned medium includes the use of a “normal” or “other” medium supplemented with a MEDII conditioned medium. The “normal” or “other” medium, such as a normal primate ES medium, can be supplemented with a MEDII conditioned medium at any concentration, but it is preferred that the “normal” or “other” medium is supplemented at between approximately 10-75%, more preferably between approximately 40-60% and most preferably approximately 50% MEDII conditioned medium. In one embodiment, the pluripotent human cell is in contact with the MEDII conditioned medium between approximately 1-60 days, more preferably between approximately 2-15 days, and most preferably 5-10 days. In a further preferred embodiment, the pluripotent cells are not passaged during the MEDII incubation.

Following MEDII incubation, cells from the embryoid body can be contacted with an essentially serum free cell differentiation medium as described above. The cells from the embryoid body can be in contact with the essentially serum free cell differentiation medium for approximately one or more days, but preferably approximately three or more days. When the cells from the embryoid body are subsequently contacted with a subsequent differentiation medium as described above, such contact can occur for approximately one or more days, but preferably approximately three or more days. The human neural cells generated using the compositions and methods of the present invention can be generated in adherent culture or as cell aggregates in suspension culture. It is to be understood that the methods of the present invention can comprise the sequential use of adherent cultures and suspension culture. Preferably, the human neural cells are produced in suspension culture.

As used herein, the term “cell differentiation environment” refers to a cell culture condition wherein the pluripotent cells or embryoid bodies derived therefrom are induced to differentiate into neural cells, or are induced to become a human cell culture enriched in neural cells. Preferably the cell lineage induced by the cell differentiation environment will be homogeneous in nature. As used herein, the term “homogeneous,” refers to a population that contains more than approximately 85%, 86%, 87%, 88%, 89%, 90%, 91%, 92%, 93%, 94%, 95%, 96%, 97%, 98%, or 99% of the desired neuronal cell lineage.

In one embodiment, the cell differentiation environment is a suspension culture where the medium is Dulbecco's Modified Eagle's Mediums and Ham's F12 media (DMEM/F12), and comprises a fibroblast growth factor (FGF) such as FGF-2. In a preferred embodiment the cell differentiation environment is a suspension culture where the medium is DMEM/F12, FGF-2, and MEDII conditioned media. In a preferred embodiment, the suspension culture is an agarose suspension culture. In one embodiment, the cell differentiation environment is also essentially free of human leukemia inhibitory factor (hLIF). In certain preferred embodiments, the cell differentiation environment is essentially serum free.

The essentially serum free cell differentiation environment can also contain supplements such as L-Glutamine, NEAA (non-essential amino acids), P/S (penicillin/streptomycin), N2 and β-mercaptoethanol (β-ME). It is contemplated that, additional factors may be added to the cell differentiation environment, including, but not limited to fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as gamma secretase inhibitors and other inhibitors of Notch processing or cleavage. Other growth factors may include members of the insulin like growth factor family (IGF), the wingless related (WNT) factor family, and the hedgehog factor family. Additional factors may be added to promote neural stem/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor.

In other embodiments, the cell differentiation environment comprises seeding the embryoid body to an adherent culture. As used herein, the terms “seeded” and “seeding” refer to any process that allows an embryoid body or a portion of an embryoid body to be grown in adherent culture. An used herein, the term “a portion” refers to at least one cell from an embryoid body, preferably between approximately 1-10 cells, more preferably between approximately 10-100 cells from an embryoid body, and more preferably still between approximately 50-1000 cells from an embryoid body. As used herein, the term “adherent culture” refers to a cell culture system whereby cells are cultured on a solid surface, which may in turn be coated with a substrate. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenascin, vitronectin, poly glycolytic acid (PGA), poly lactic acid (PLA), poly lactic-glycolic acid (PLGA) and feeder cell layers such as, but not limited to, primary astrocytes, astrocyte cell lines, glial cell lines, bone marrow stromal cells, primary fibroblasts or fibroblast cells lines. In addition, primary astrocyte/glial cells or cell lines derived from particular regions of the developing or adult brain or spinal cord including but not limited to olfactory bulb, neocortex, hippocampus, basal telencephalon/striatum, midbrain/mesencephalon, substantia nigra, cerebellum or hindbrain may be used to enhance the development of specific neural cell sub-lineages and neural phenotypes. Furthermore, the substrate for the adherent culture may comprise the extracellular matrix laid down by a feeder cell layer, or laid down by the pluripotent human cell or cell culture.

The human neural cells produced using the methods of the present invention have a variety of uses. In particular, the neural cells can be used as a source of nuclear material for nuclear transfer techniques, and used to produce cells, tissues or components of organs for transplant. The neural cells of the present invention can also be used in human cell therapy or human gene therapy to treat neuronal diseases such as Parkinson's disease, Huntington's disease, lysosomal storage diseases, multiple sclerosis, memory and behavioral disorders, Alzheimer's disease and macular degeneration. Other pathological conditions including stroke and spinal cord injury can be treated using the neural cells of the present invention. The neural cells can also be used in testing the effect of molecules on neural differentiation or survival, in toxicity testing or in testing molecules for their effects on neural or neuronal functions. This could include screens to identify factors with specific properties affecting neural or neuronal differentiation, development, survival or function. In this application the cell cultures could have great utility in the discovery, development and testing of new drugs and compounds that interact with and affect the biology of neural stem cells, neural progenitors or differentiated neural or neuronal cell types.

The neural cell or the human cell culture enriched in neural cells may disperse and differentiate in vivo following brain implantation. In particular, following intraventricular implantation, the cell can be capable of dispersing widely along the ventricle walls and moving to the sub-ependymal layer. The cell can be further able to move into deeper regions of the brain, including into the untreated (e.g., by injection) side of the brain into sites that include the thalamus, frontal cortex, caudate putamen and colliculus. In addition the neural cell or human cell culture enriched in neural cells can be injected directly into neural tissue with subsequent dispersal of the cells from the site of injection. This could include any region, nucleus, plexus, ganglion or structure of the central or peripheral nervous systems.

The invention further provides methods of producing a partially differentiated pluripotent cell comprising culturing pluripotent cells on a layer of fresh feeder cells, wherein the fresh feeder cells are less than 2 days old, thereby inducing formation of a more differentiated pluripotent cell. In preferred embodiments, the fresh feeder cells are less than one day old, more preferably less than 12 hours old, or more preferably less than 6 hours old. In preferred embodiments, the more differentiated pluripotent cell is obtained from the central, or crater, region of the colony of pluripotent cells. In some embodiments, the more differentiated pluripotent cell expresses less Oct4 marker than an embryonic stem cell. The invention further provides a composition comprising these more differentiated pluripotent cells.

Throughout this application, various publications are referenced. The disclosures of all of these publications and those references cited within those publications in their entireties are hereby incorporated by reference into this application in order to more fully describe the state of the art to which this invention pertains. In particular, U.S. Provisional Patent Application Nos. 60/401,968 and 60/459,090, and International Application PCT/AU03/00552 are hereby incorporated by reference in their entirety. The following examples are not intended to limit the scope of the claims to the invention, but are rather intended to be exemplary of certain embodiments.

### EXAMPLES

### Example 1

Production of Essentially Serum Free MEDII Conditioned Media

An essentially serum free MEDII conditioned medium was produced as follows. Hep G2 cells (Knowles et al., 1980 Nature 288:615-618; ATCC HB-8065) were seeded at a density of 5×104 cells/cm2 and cultured for three days in DMEM. Cells were washed twice with 1×PBS and once with serum free medium (DMEM containing high glucose but without phenol red, supplemented with 1 mM L-glutamine, 0.1 mM β-ME, 1×ITSS supplement (Boehringer Mannheim), 10 mM HEPES, pH 7.4 and 110 mg/L sodium pyruvate) for 2 hours. Fresh serum free medium was added at a ratio of 0.23 ml/cm2 and the cells were cultured for a further 3-4 days. SfMEDII was collected, sterilized and stored. A further explanation of MEDII conditioned media can be found in International Application No. WO 99/53021, herein incorporated by reference in its entirety.

### Example 2

Isolation of the Neural Inducing Component of MEDII Media

Essentially serum free MEDII (sfMEDII) or serum containing MEDII is used as a source of the biologically active factor in all purification protocols in this examples. The bioactive component of MEDII, referred to as the neural inducing factor, is routinely isolated from the sfMEDII or MEDII using purification techniques well known in the art. These techniques can include ultrafiltration; column chromatography, including ion exchange columns, hydrophobic columns, hydroxyapatite and gel-filtration columns; affinity chromatography; high performance liquid chromatography (HPLC); or FPLC. After each step of the purification protocol, individual samples are assayed directly for the biological activity of the neural inducing factor on ES cells. Reducing SDS PAGE can reveal the presence of a highly purified component in samples containing the neural inducing factor bioactivity.

The purified and isolated neural inducing component is capable of indefinite storage, and is used in place of MEDII conditioned medium to induce the formation of neural cells from pluripotent or multipotent stem cells, or to enrich a human cell culture for neural cells.

### Example 3

Derivation and Characterization of a Neurosphere Population from Mouse Embryonic Stem Cells

Preparation of Embryoid Bodies from Mouse ES Cells

D3 mouse embryonic stem cells were maintained on gelatin free tissue culture plates and passaged every three to four days. The mouse ES cell culture medium was 10% fetal bovine serum (FBS), Dulbecco's Modified Eagles Medium (DMEM), 0.1M β-mercaptoethanol (β-ME), 1 mM glutamine, 1000 U/ml mouse LIF (ESGRO). The ES cell colonies were rinsed twice with PBS and treated with Trypsin/EDTA for one minute, they were then triturated and blocked with an equal volume of FBS, and then centrifuged, resuspended and counted.

Embryoid bodies were formed by seeding the ES single cell suspension at 1×10⁵ cells/ml in IC:DMEM media (10% FBS, 90% DMEM, 1 mM Glutamine, and 0.1% β-ME). Embryoid bodies were allowed to aggregate for two days, and were then split 1:2 in IC:DMEM media (EB²), cultured for a further 2 days and split again 1:2 (EB⁴) and then cultured for three further days with daily changes of media (EB⁷). The culture conditions were then changed to essentially Serum-Free media (50% DMEM, 50% Ham's F12 (Gibco BRL), 1×ITSS (Boehringer-Mannheim) and 10 ng/ml FGF-2 (Peprotech Inc.) for a further eight days (EB¹⁵).

To produce MEDII treated embryoid bodies (EBMs), a single cell suspension of ES cells was seeded at 1×10⁵ cells/ml in IC:DMEM supplemented with MEDII (50% MEDII). The MEDII conditioned medium used in the experiments described in this Example was produced in the same manner as the essentially serum free MEDII conditioned medium described in Example 1, except that it contained 5% fetal calf serum. EBMs were allowed to aggregate for two days and then were split 1:2 in 50% MEDII media (EBM²), cultured for a further 2 days and split 1:2 (EBM⁴, EPL cells in suspension and then cultured for three further days with daily changes of 50% MEDII media (EBM⁷). After day seven, the medium was then changed to Serum-Free medium as it was for EBs for a further eight days (EBM¹⁵).

All-Trans Retinoic Acid Supplementation

For treatments involving all-trans retinoic acid (RA), a 4−/4+ culture supplementation method was used. Briefly, both EB and EBM cell aggregates were allowed to form in appropriate media for four days, and the media was then supplemented with 100 nM all-trans retinoic acid (Sigma) with each daily media change thereafter for a further four days.

Generation of Neurosphere Suspension Cultures

Both EBs and EBMs were triturated to a near single cell suspension after being cultured for periods of 7, 9, 12, and 15 days (7 days in MEDII followed by an appropriate number of days in essentially Serum-Free media). Two methods of trituration, either mechanical dissociation or trypsin dissociation, yielded similar results. Cells were seeded at approximately 10-20 cells/μl of media into 10 mls of neurosphere media (DMEM:F12, 10 ng/ml FGF-2, 10 μg/ml heparin (SIGMA), 1/50 B27 (Gibco BRL), 1/100 penicillin/streptomycin, 1/100 ITSS) in a T75 culture flask. Cultures were maintained in this media with a 50:50 change of fresh media after 7 days and cultured for 10-12 days. Sphere formation was readily apparent after three days in culture, and robust spheres had formed by day 7. One population of spheres derived from EBM¹² (no RA) aggregates was passaged and grown for a further two passages to yield tertiary spheres.

The tertiary spheres were seeded onto Poly-L-Ornithine/Fibronectin coated chambers slides (Nunc) and cultured in neurosphere medium for three days before fixing and processing for three representative neuronal lineage immunohistochemical markers, NF200, O4, and GFAP.

Grafts into the Rat Striatum

To prevent graft rejection of implanted mouse cells rats received daily immunosuppression by i.p. injection of cyclosporin A diluted in extra virgin olive oil (1 mg/kg Sandimmune, Sandoz Pharmaceutical). Daily injections started one day prior to the implantation of cells. Rats were intubated and anesthesia was maintained (Ventilator 74 strokes/min, 2 ml strokes, halothane 1-1.5%, oxygen flow rate at less than 1 liter/min). Animals were mounted in a Kopf Stereotaxic frame (Kopf Instruments, Tujanga, Calif.), the head was shaved and Betadine applied to the shaven area. An incision was made to expose bregma and lambda and the nose bar adjusted to level bregma and lambda. Each injected animal received a 1 μl volume (1000-2000 cells/μl) delivered over 1 minute into the right striatum using a 5 μl SGE positive displacement syringe and a 23 G needle (SGE International, Victoria, Australia). Striatal coordinates were: anterior 0.9 mm, lateral 2.7 mm, depth 5.0 mm. The needle was left in place for 4 minutes and removed over a one minute period. The rat was sutured and Xylocaine and Ilium Dermapred were applied to the suture line. The intubation tube was removed and the rat was allowed to recover on a heating pad. A subcutaneous injection of butorphanol (2 ml/kg) was administered as a post-operative analgesic. Engrafted rats were left for a period of 4 weeks and brains were harvested by transcardial perfusion with PBS and 4% PFA, dissecting away the skull. The brains were stored in PBS containing 0.01% sodium azide.

Results

Formation of neurospheres differed dramatically between single cell suspensions generated from either ES cell aggregates grown in either IC:DMEM (EBs) or grown in 50% MEDII (EBMs). From Table 1, it is shown that ES cell aggregates formed in IC:DMEM followed by periods of serum starvation exhibit poor sphere forming capacity even when treated with the potent neural inducer, all-trans retinoic acid. In contrast, a MEDII dependent effect was observed in cell aggregates that had formed in 50% MEDII followed by a period of serum starvation. Robust sphere forming capacity was clearly seen in EBM¹² aggregates with sphere formation visible after 34 days in neurosphere culture media. The capacity for sphere formation seemed to be diminished in EBMs on either side of this time frame. An effect of all-trans retinoic acid was observed such that sphere forming capacity seemed to emerge earlier at EBM⁹, and show a decrease with further culturing. In both cases, robust sphere forming capacity was only seen in cells derived from embryoid bodies that had been conditioned in 50% MEDII.

TABLE 1
Neurosphere formation is dependent on MEDII, and is influenced by retinoic acid.

| | | Day 9 | Day 12 | Day 15 |
|---|---|---|---|---|
| | | (50% MEDII or | (50% MEDII or | (50% MEDII or |
| | Day 7 | IC:DMEM plus | IC:DMEM plus | IC:DMEM plus |
| | (50% MEDII or | 2 days essentially | 5 days essentially | 8 days essentially |
| | IC:DMEM only) | serum free) | serum free) | serum free) |
| EB | − | − | − | − |
| EB + RA | − | − | − | − |
| EBM | + | + | +++ | ++ |
| EBM + RA | + | +++ | + | + |

EB = embryoid body;
EBM = embryoid body cultures in 50% MEDII;
RA = 100 nM all-trans retinoic acid.
“−“ indicates no or very poor sphere formation,
“+” indicates poor sphere forming capacity,
“++” indicates moderate sphere forming capacity, and
“+++” indicates high sphere forming capacity.

Sphere formation was observed after two further passages of EBM¹² (no RA) spheres that were mechanically passaged and reseeded at a 10-20 cells/μl density in; neurosphere media. During the passaging of these cells it was noted that dense networks of cells formed on the bottom of the flask where sphere had attached. These networks exhibited extensive neural morphology and extensive networks of neurites were observed. Dense clusters of cells appeared and were likely to act as a source of more spheres. Spheres from these tertiary passaged spheres were seeded onto glass chamber slides and allowed to grow for three days before processing for immunohistochemistry. These single spheres grew to form similar extensive networks of cells with dense regions that appeared to be forming more spheres. Marker analysis revealed that there were large numbers of GFAP positive astrocytic lineage cells, moderate numbers of NF200 positive neurons, and low levels of 04 positive oligodendrocytes. The seeded spheres were therefore capable of producing cells of all three neural lineages after three passages at clonal cell densities and therefore provide evidence of self-renewal and multi-potency.

An in vivo analysis of low cell number grafts into the rat Striatum revealed detectable mouse cells that line the needle tract of the injection site. No obvious signs of gross teratoma formation were visible and the number of detectable cells was low (10-20 per 10 μM section through the graft site).

### Example 4

Derivation and Characterization of a Neurosphere Population from Mouse Embryonic Stem Cells Using a Two-Stage Process

Example 3 was generally repeated, however, a two-stage process was followed with cells grown as aggregates/embryoid bodies in the one media (Stage A) prior to disaggregation for Stage B growth conditions. Mouse ES cells were separated by treatment with trypsin and the single ES cell suspension seeded in a cellular aggregate culture media (DMEM:F12 and either N2 or ITSS) that was free of serum. The addition of 10 ng/ml FGF2 and/or 100 nM dose of RA is optional. Alternatively the cell aggregates/embryoid bodies are formed in the presence of DMEM and 100 μM proline with either N2 or ITSS and optionally with FGF2 and RA. Cell aggregates/embryoid bodies were allowed to form in Costar low attachment tissue culture dishes for a period of up to 15 days in suspension culture (Stage A) and then were triturated to a single cell suspension using trypsin dissociation.

Cells were then seeded at a concentration of less than 100 cells/μL in neurosphere media (DMEM:F12, 10 ng/ml FGF2, 10 μg/ml heparin (SIGMA), 1/50 B27 (GIBCO), 1/100 pen/strep, 1/100 ITSS) in a T75 culture flask. Cultures were maintained in this media for 14 to 21 days (Stage B) and the neurospheres so formed can be maintained by passaging in the same media.

These cell aggregates tended to demonstrate an irregular appearance of the cell layer. Robust embryoid body formation with morphological characteristics of neurectoderm did not occur without the presence of F12 media.

The neurospheres formed can be seeded onto poly-L-ornithine/laminin coated plates and allowed to adhere and differentiate. Optionally in this culture stage, neurospheres are maintained in media containing combinations of RA, 50% MEDII and L-proline. In an alternative treatment, during stage A, the embryoid bodies (EB9) are seeded onto poly-L-ornithine/laminin coated culture plates and cultured for 6 to 8 days to permit neuronal differentiation.

In an alternative treatment, embryoid bodies cultured from stage A are triturated and resuspended in a minimal media (DMEM/F12 and N2 or ITSS). Optionally, this media includes combinations of FGF, MEDII, RA and L-proline. The aggregates formed can also be seeded onto poly-L-ornithine/laminin coated plates and allowed to adhere and differentiate.

### Example 5

Formation and Characterization of EBs from Human ES Cells in Essentially Serum Free Conditions

Manual Passaging of Human ES Cells

Human embryonic stem cells (HESCs) identified as BGN01 and BGN02 (BresaGen, Inc. Athens, Ga.) were used in this work. The HESCs were grown in DMEM/F12 (50/50) supplemented with 15% FBS, 5% knockout serum replacer (Invitrogen), 1× non-essential amino acids (Invitrogen), L-Glutamine (20 mM), penicillin (0.5 U/ml), streptomycin (0.5 U/ml), human LIF (10 ng/ml, Chemicon) and FGF-2 (4 ng/ml, Sigma). The human ES cells were grown on feeder layers of mouse primary embryonic fibroblasts that were mitotically inactivated by treatment with mitomycin-C. Feeder cells were re-plated at 1.2×10⁶ cells per 35 mm dish. The mitotically inactivated fibroblasts were cultured for at least 2 days prior to the plating of HESCs. The HESCS were manually passaged onto fresh fibroblast feeder layers every 3-4 days using a fire-pulled Pasteur pipette. Briefly, the barrel of the Pasteur pipette was melted solid and drawn out to a solid needle approximately 1 cm long and approximately 25 μm in diameter, which was sequentially pressed through HESC colonies to form a uniform grid of cuts. The same needle was passed under the colonies to lift them from the feeder layer. Entire plates of HESCs were harvested, then the colonies were broken into individual pieces defined by the grid by gentle pipetting using a 5 ml serological pipette. The pieces from a single plate were split between 2 or 3 new plates that were coated with feeder layers of mitotically inactivated mouse primary embryonic fibroblasts.

SSEA4 Selection and Bulk Passaging of HESCs

SSEA4 staining appears to be closely associated with the undifferentiated state of HESCs. Undifferentiated domed HESC colonies show a uniform distribution of SSEA4 immunostaining, while differentiating HESC colonies show reduced or no expression of SSEA4 in morphologically differentiated cells. An example of this is the reduced SSEA4 expression in morphologically differentiated cells that occurs within the crater cells located in the center of manually passaged HESCs that are plated onto fresh feeder layers These crater cells grow as a monolayer, surrounded by multilayered morphologically undifferentiated HESCs. Since SSEA4 appears to be selective for a population of undifferentiated HESCs, it was chosen to use as a selectable marker.

Undifferentiated HESCs were selected by magnetic sorting using an anti-SSEA4 antibody (Developmental Studies Hybridoma Bank) and the MACS separation system (Miltenyi Biotec) according to the manufacturers instructions. Briefly, manually passaged HESCs were harvested by treating with 1 mg/ml Collagenase (Gibco) for 5 minutes, followed by treating with 0.05% Trypsin/EDTA for 30 seconds. Colonies were then flushed off the top of the feeder layer and dissociated to an essentially single cell suspension, leaving the feeder cells behind as a net. The trypsin was neutralized with 10% FBS/10% KSR human ES medium and passed through a cell strainer (Becton Dickinson). For blocking, cells were pelleted and resuspended in staining buffer (5% FBS, 1 mM EDTA, penicillin (0.5 U/ml) and streptomycin (0.5 U/ml), in Ca2⁺/Mg2⁺ free PBS).

The cells were pelleted and resuspended in 1 ml primary anti-SSEA4 antibody diluted 1:10 in staining buffer, and incubated at 4° C. for 15 minutes. 9 ml staining buffer was then added and the cells were pelleted, washed with 10 ml staining buffer and re-pelleted. 1×10⁷ cells were resuspended in 80 μl staining buffer and 20 μl magnetic goat anti-mouse IgG MicroBeads were added, mixed and incubated at 4° C. for 10 minutes. The volume was then brought to 2 ml with staining buffer and 2 μl of a fluorescent conjugated secondary antibody (Alexa-488 conjugated goat anti-mouse IgG, Molecular Probes) was added to enable fluorescent analysis of the separation. The sample was incubated for 5 minutes at 4° C., then the volume was brought to 10 ml with staining buffer and the cells were pelleted and washed in 10 ml staining buffer and re-pelleted. The cells were resuspended in 500 μl staining buffer and applied to a separation column that had been prepared by washing it three times with 500 μl staining buffer. The column was positioned on the selection magnet prior to application of the cells and the flow-through and three washes with 500 μl staining buffer were collected. These cells in these fractions were presumably a SSEA4 negative population. The column was removed from the magnet, 500 μl staining buffer was added and forced through with a plunger, and the presumed SSEA4 positive cell population was collected in a 15 ml tube. 20% KSR human ES growth medium was added to bring the volume to 10 ml, and the cells were pelleted and resuspended in 1 ml of the same medium. 10⁵ SSEA4 selected HESCs were plated on 35 mm dishes plated with a mouse embryonic fibroblast feeder layer, and the cells were maintained and passaged in 20% KSR growth medium (see below).

To examine the effectiveness of the selection, aliquots of the flow/wash sample and SSEA4 selected sample were analyzed by fluorescence microscopy. Approximately 75% of the cells from the retained fraction were SSEA4 positive, indicating effective enrichment.

Bulk passaged HESCs were grown in DMEM/F12 (50/50) supplemented with 20% knockout serum replacer (KSR; Invitrogen), 1×NEAA (Invitrogen), L-Glutamine (20 mM), penicillin (0.5 U/ml), streptomycin (0.5 U/ml), human LIF (10 ng/ml, Chemicon) and FGF-2 (4 ng/ml, Sigma). For passaging, cells were treated with 1 mg/ml Collagenase (Gibco) for 5 minutes, followed by 0.05% Trypsin for 30 seconds and the cells were then dissociated with a 1 ml pipette. The feeder layer remained as a mesh and was removed with a pipette. DMEM/F12 (50/50) supplemented with 10% FBS and 10% KSR was added to the HESC suspension, followed by centrifugation, aspiration and resuspension in culture medium. HESCs were replated at 1×10⁵ cells per 35 mm dish on a feeder layer.

Formation of Essentially Serum Free Embryoid Bodies

HESC cultures were washed once with DMEM/F12 and once with DMEM/F12 supplemented with 1×N2 supplement (Invitrogen). Undifferentiated HESC colonies were harvested by the manual passaging methods described above into uniform colony pieces of approximately 10-100 cells. Pieces were transferred to 15 ml tubes and washed in 10 ml DMEM/F12 plus 1×N2 supplement. The pieces were left to settle, and the medium was aspirated. The pieces were resuspended in 2.5 ml of medium, and transferred to suspension dishes.

Suspension dishes were prepared by coating the surface of non-tissue culture plastic Petri dishes with a layer of agarose. The agarose coating was generated by pouring a molten solution of 0.5% agarose in DMEM/F12 medium into the Petri plates. The agarose coating was equilibrated in DMEM/F12 medium. Suspension cultures contained 2.5 ml of medium for 35 mm dishes, or 10 ml of medium for 100 mm dishes.

Essentially serum free embryoid bodies were cultured in suspension for up to four weeks, with replenishment of the medium every 3-4 days. The essentially serum free embryoid bodies were passaged every 5-7 days by cutting them into pieces with drawn out solid glass needles. At passaging, the embryoid bodies contained approximately 5000-10,000 cells and were divided into 4-10 pieces. Essentially serum free embryoid bodies formed in the presence of DMEM/F12 with 1×N2 and FGF-2 are termed sfEBs, while essentially serum free embryoid bodies formed in the presence of DMEM/F12 with 1×N2 and FGF-2 and 50% MEDII are termed sfEBMs.

Clonal Derivation of Colonies from Essentially Serum Free Embryoid Bodies Treated with MEDII Medium

sfEBMs were transferred to tissue culture dishes (4-well or 35 mm) and were rinsed with Ca²⁺/Mg²⁺ free PBS. After settling, the PBS was aspirated and 1 or 2 ml of 0.25% trypsin was added. sfEBMs were dispersed to a single cell suspension, with occasional doublets or triplets, by gentle trituration using a 1 ml pipette. The cell suspension was transferred to a 15 ml tube, diluted with 9 or 18 ml of medium and pelleted by centrifugation. The pellet was resuspended in DMEM/F12 supplemented with 1×N2 supplement, FGF-2 (4 ng/ml), 50% MEDII and added to agarose coated suspension dishes at low density (0.5-5 cells/μl). These cultures were not disturbed for 4-5 days, when small uniform colonies could be observed. Colonies could be harvested at approximately 10 days.

Immunohistochemistry

Essentially serum free embryoid bodies (sfEBs and sfEBMs) were cut into pieces using glass needles and 1-15 pieces were plated onto polyornithine/laminin coated permanox chamber slides in the same medium used for suspension culture. Polyornithine/laminin coated slides were prepared by diluting polyornithine to 20 μg/ml in tissue culture grade water, coating chamber wells at 37° C. overnight, washing twice with water and coating the chamber wells with 1 μg/ml laminin at 37° C. overnight. The slides were washed with water and 1×PBS prior to plating the cells. The embryoid bodies were cultured on these slides for 2-7 days.

For preparing cytospins, embryoid bodies in suspension were disaggregated and attached to a glass slide using a standard cytospin approach for immunostaining (Watson P. A., J. Lab. Clin. Med. 68:494-501, 1966). sfEBMs were washed with 1×PBS and disaggregated with 0.05% trypsin and gentle trituration. The cell suspension was washed with culture medium, pelleted and resuspended in HESC medium and 1×104 cells were attached to a glass microscope slide by centrifugation at 300 g for 4 minutes using a cytospin apparatus (Heraeus Instruments GmbH). The attached cells were fixed immediately with 4% paraformaldehyde, and 4% sucrose in 1×PBS for 15 minutes, followed by three separate 5-minute washes in 1×PBS.

For immunostaining, on fixed cells or cytospins, the samples were rinsed with 1×PBS and fixed in 4% paraformaldehyde, 4% sucrose in 1×PBS for 30 minutes at 4° C. The cells were then washed in 1×PBS and stored at 4° C. To perform immunostaining, the cells were washed in block buffer (3% goat serum, 1% polyvinyl Pyrolidone, 0.3% Triton X-100 in wash buffer) for 30 minutes, and then incubated with the appropriate dilution of the primary antibody, or combination of antibodies for 4-6 hours at room temperature. The primary antibodies were anti-Map2, a mouse monoclonal antibody recognizing the Map-2 a, b and c isoforms (Sigma, Catalog # M4403) at a 1/500 dilution; anti-Sox1, a chicken monospecific polyclonal antibody (Chemicon, Catalog # AB5934) at a 1/250 dilution; and anti-Nestin, a rabbit polyclonal antibody (Chemicon, Catalog # AB5922) at a 1/200 dilution; anti-Oct4, a rabbit polyclonal antibody (Santa Cruz, Catalog # sc-9081) at a 1/200 dilution; sheep anti-Tyrosine Hydroxlyase (TH) antibody (Pel-Freez, Catalog # P60101-0) at a 1/500 dilution; anti-SSEA4, a mouse monoclonal antibody (Developmental Studies Hybridoma Bank, Catalog # MC-813-70) at a 1/5 dilution.

The cells were then washed in wash buffer (50 mM Tris-HCL pH 7.5, and 2.5 mM NaCl; 3 times for 5 minutes each). The cells were then incubated for a minimum of 2 hours in secondary antibodies diluted 1:1000, followed by washing in wash buffer. The secondary antibodies were Alexa-350 (blue), 488 (green) or 568 (red) conjugated goat anti-chicken, anti-rabbit, or anti-mouse antibodies, all available from Molecular Probes. The cells were stained with 5 ng/ml DAPI to detect cell nuclei, and were then washed from overnight to 2 days in a large volume of wash buffer. The slides were mounted with mounting medium and a cover slip. Slides were visualized using a either a NIKON TS100 inverted microscope or a NIKON TE 2000-S inverted microscope with a Q Imaging digital camera.

Results

Human embryonic stem cell colony pieces grown in suspension culture in essentially serum free medium containing FGF-2, with or without MEDII conditioned medium, became spherical within 6 hours of derivation, and developed into essentially uniform spherical suspension cultures. The presence of MEDII appeared to be beneficial to overall morphology and cell viability during the early derivation of essentially serum free embryoid bodies. sfEBMs exhibited reduced cell death in suspension culture and improved overall morphology in comparison to sfEBs in the first three days after derivation. In general, sfEBM exhibited a faster growth rate than sfEBs.

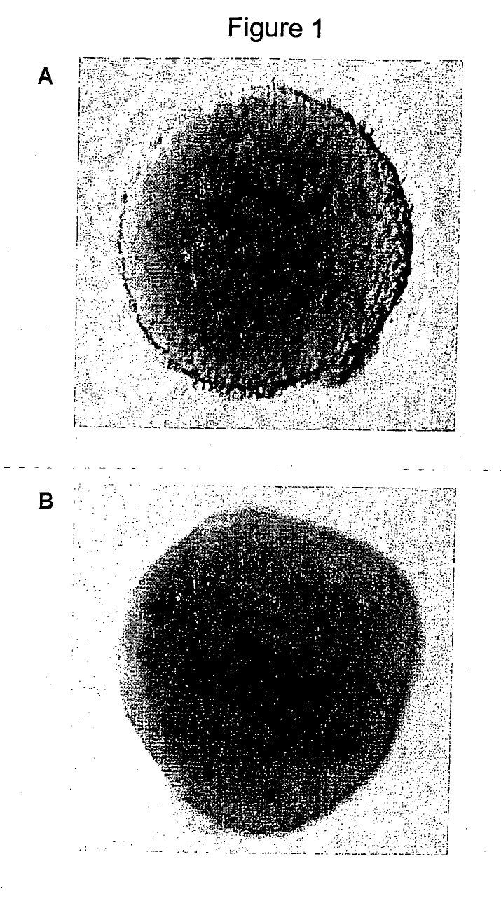

After one week of culture, two distinct types of essentially serum free embryoid bodies could be observed: a solid type, and a structured type. FIGS. 1A and 1B show derived serum free embryoid bodies. HESC colonies were dissected into uniform sized pieces and cultured in suspension. Solid essentially serum free embryoid bodies (FIG. 1A) were distinguished from structured essentially serum free embryoid bodies (FIG. 1B) after one week. Solid essentially serum free embryoid bodies appeared highly uniform, and did not exhibit any obvious differentiation such as endoderm formation, or cavitation. Structured essentially serum free embryoid bodies developed a morphology that appeared to be comprised of multiple spherical compartments, each of which may, or may not, have contained an inner cavity. These appeared to be three dimensional spherical equivalents of the cell rosettes observed during subsequent adherent culture. Structured essentially serum free embryoid bodies also did not exhibit differentiation of an endoderm layer. Solid and structured embryoid bodies were observed with and without the presence of MEDII. In some instances a single embryoid body exhibited both types of morphology as distinct regions.

To examine neural differentiation, essentially serum free embryoid bodies were seeded onto polyornithine/laminin coated slides and were allowed to develop in adherent culture in essentially serum free medium. Both sfEBs and sfEBMs adhered, flattened somewhat and exhibited outgrowths of cells in culture periods of up to one week. The structured sfEBs and sfEBMs could undergo proliferation and developed distinctive cellular organization to radial rosette structures, a characteristic of neural stem/progenitor cells.

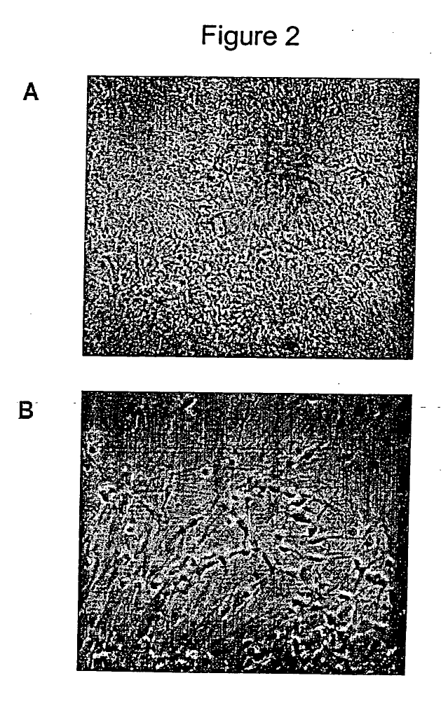

FIGS. 2A-2B show neural differentiation of serum free embryoid bodies. FIG. 2A shows rosette proliferation observed in sfEBs seeded in the presence of 50% MEDII. FIG. 2B shows neuron network differentiation observed in sfEBs seeded in the presence of 50% MEDII. Some of the structured essentially serum free embryoid bodies developed outgrowths that appeared to be essentially pure populations of rosettes. While proliferation and development of rosettes were observed in the absence of MEDII, they were far more extensive in the presence of MEDII. MEDII thus has an obvious positive effect on the formation of neural cell types.

Structured essentially serum free embryoid bodies exhibited extensive differentiation of neurons in the presence of MEDII, with large neuron networks observed. A markedly reduced level of neuron differentiation was observed in adherent culture in the absence of MEDII. The structured essentially serum free embryoid bodies contained rosettes consisting of relatively pure populations of neural precursors (Sox1⁺ cells), and partially differentiated neural precursors (cells co-expressing Sox1 and a radial pattern of Map2 staining). These precursor cell types were tightly packed with nuclei distributed throughout the rosettes. Precursor cells were also closely associated with networks of differentiated neurons (Map2⁺ cells) at their periphery. Rosette cells also co-expressed Nestin, a neural precursor marker, with the radial Map2 expression. The radial pattern of Map2 expression was clearly different from the intense dendrite specific Map2 staining associated with differentiated and more mature neurites. Differentiated neurons were observed in close association with or emanating from the rosettes, and were presumed to differentiate from these precursor cells.

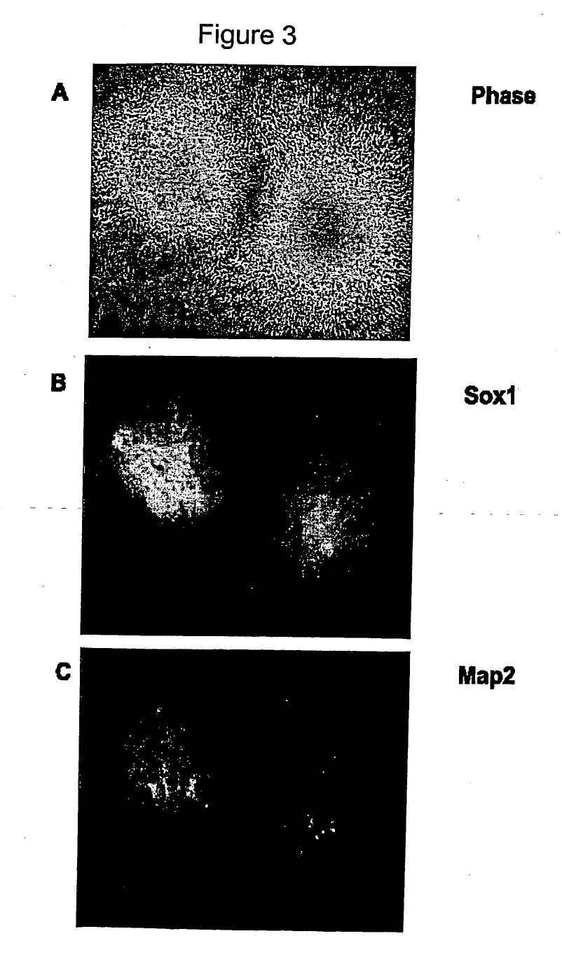

Staining of the structured essentially serum free embryoid bodies with anti-Nestin, anti-Sox1, and anti-Map2 further demonstrated the presence of multiple neural cells types: neural progenitors (Nestin⁺/Sox1⁺, and/or semi-differentiated neurons (Nestin⁺/Sox1⁺/radial Map2⁺), differentiated neurons (Map2⁺), and presumptive glial cells (Nestin⁺). FIGS. 3A-3C show serum free embryoid bodies containing neural precursors. FIG. 3A shows phase contrast micrograph of seeded structured (left) and solid (right) serum free embryoid bodies. Greyscale images of fluorescent stainings with anti-Sox1 is shown in FIG. 3B and anti-Map2 antibodies is shown in FIG. 3C. Solid serum free embryoid bodies contain a high proportion of neural precursors (Sox1⁺ cells) and rare neurons (Map2⁺). Structured serum free embryoid bodies contain rosettes of semi-differentiated neural precursors (Sox1⁺/Map2⁺) and neurons (Map2⁺).

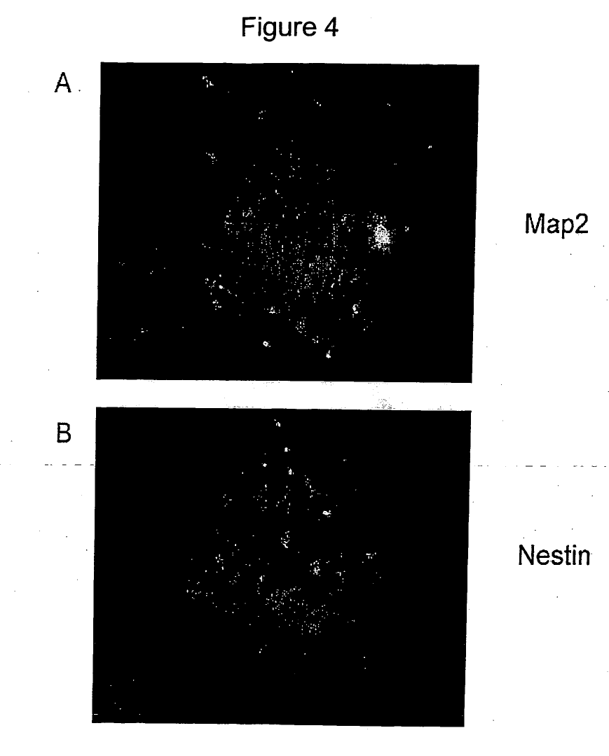

FIGS. 4A-4B show Map2 and nestin expression in rosettes. Radial Map2⁺ and nestin⁺ expression indicates a semi-differentiated progenitor/young neuron.

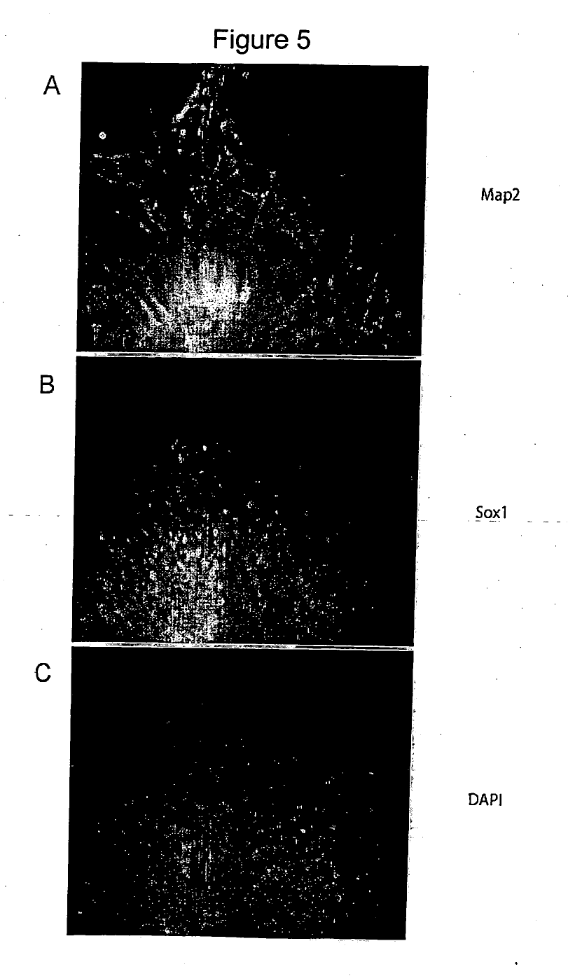

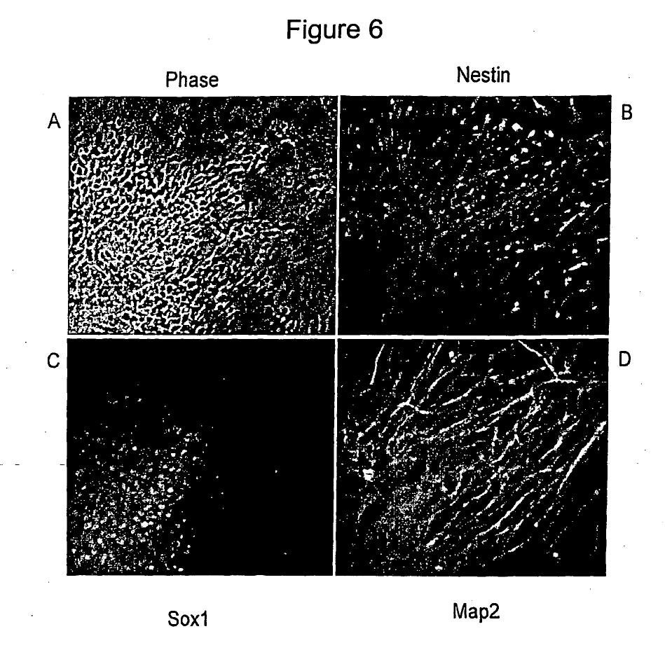

FIGS. 5A-5C show Sox1, Map2, and DAPI expression in serum free embryoid body differentiation. Map2 expression in FIG. 5A indicates a semi-differentiated neuron (radial pattern) and differentiated neurons (network, dendritic stain), Sox1 expression in FIG. 5B indicates neural precursors, and DAPI in FIG. 5C is a DNA stain highlighting all nuclei in the field. Virtually all nuclei in the rosette are double stained for Sox1/DAPI, indicating a relatively pure population of neural precursors/progenitors (Sox1⁺) and/or semi-differentiated neural precursors/progenitors (Sox1⁺/Map2⁺).

Solid essentially serum free embryoid bodies also exhibited extensive proliferation, but did not develop rosettes or exhibit neurite networks. These outgrowth cultures were analyzed by immunocytochemistry for neural markers. Staining with anti-Sox1 and anti-Map2 antibodies, which identify neural precursors and neurons respectively, demonstrated highly efficient neural differentiation in these cultures. Solid essentially serum free embryoid bodies contained a high proportion of neural precursors (Sox1⁺ cells) and relatively few differentiated neurons (Map2⁺ cells).

Essentially serum free embryoid bodies could be maintained and were stable through multiple passages in suspension culture. MEDII appeared to enable more rapid proliferation of essentially serum free embryoid bodies over extended culture in suspension.

In an attempt to derive clones of essentially serum free embryoid bodies, trypsin was used to generate single cell suspensions from sfEBM cultures and low density cell suspensions, from 0-0.5-5 cells/μl, were incubated in suspension dishes. Small uniform colonies were observed at 4-5 days in essentially serum free medium that contained FGF-2 (4 ng/ml) and 50% MEDII. No clonal colonies could be generated in medium that contained FGF-2 or MEDII alone. The synergistic activities of FGF2 and MEDII in the derivation of clonal sfEBMs was unexpected. Titration of the MEDII revealed that this portion of the colony derivation activity could function when diluted down to 10% of the medium. The colonies produced were presumed to be clonal, and proliferated to as much as 0.2 mm in diameter within 10 days. The cloning efficiency was in the order of 4.5-6%.

Cloned essentially serum free embryoid bodies passage 1 (sfEBMc1) were similar to solid essentially serum free embryoid bodies in overall morphology, although the sfEBMc1s were highly sticky, and were able to adhere to and develop colonies on agarose coated plates. After a week in culture, some colonies detached and developed in suspension and exhibited a morphology very similar to solid essentially serum free embryoid bodies. When seeded on polyornithine/laminin coated slides, sfEBMc1 developed highly homogeneous outgrowths. The outgrowth cells grew as a radial monolayer and were not obviously neural precursors or neurites, but did exhibit many cellular extensions, and appeared to be glial-like in morphology. No rosettes were observed. Staining with anti-Map2 revealed that these colonies retained some capacity for neuronal differentiation, although this was markedly reduced when compared to solid essentially serum free embryoid bodies, with only rare neurons or very small neuronal networks being observed. SfEBMc1 cultures did not exhibit any colonies similar to structured essentially serum free embryoid bodies.

Passaging from sfEBMc1 colonies to sfEBMc2 cultures was possible using the same trypsin based approach, although the overall proliferation rate of sfEBMc2 colonies appeared to be slower than in sfEBMc1 cultures.

### Example 6

Method to Derive Neuralized Serum Free EBM from HESC Crater Colonies

The colony morphology of HESCs differed from the typical multilayered, domed colonies when HESCs were plated onto feeder cells that had been freshly plated. When plated on feeder cells that were 0-6 hours old, but not on feeders that were 2 days old or older, typical HESC colonies formed except that in the central region of the colony a “crater” was observed. These central cells formed a monolayer of uniform cells, within a ring of multilayered HESCs. This monolayer was in direct contact with the tissue culture plastic, or the extracellular matrix that was left behind as the HESC colony had pushed out the underlying feeder layer. HESC colonies typically displace the underlying feeder layer as they seed and proliferate. Cells within the crater expressed the pluripotent marker Oct-4, although apparently at a reduced level compared to the surrounding ring of HESCs, indicating that they are a novel, partially differentiated cell type derived from the HESCs. This approach allowing the controlled development of crater HESC colonies occurred within 3 to 5 days and generated a uniform monolayer of central cells, as opposed to stochastic differentiation proceeding over several weeks and leading to a complex heterogeneous culture.

The differentiation capacity of the crater cells was tested in the serum free embryoid body system. Crater cells were purified by removing the feeder layer and HESC growing on their surface. Watchmakers forceps were used to hold the feeder layer at the side of the culture dish, and lift this layer and attached multilayered HESC from the dish. This manipulation peels the feeder layer and the multilayered parts of the HESC colonies off of the dish and leaves behind the cells that had formed the crater. The monolayer crater cells were left attached to the dish. Glass needles were used to cut the crater monolayer to 50-200 cell size pieces, and lift them from the dish. These pieces were grown in suspension culture in same essentially serum free conditions as in Example 4 (DMEM/F12/N2/FGF-2 (4 ng/ml)/with or without 50% MEDII). After 4 to 5 days suspension culture, serum free EB formed in this way exhibited structured regions morphologically similar to those seen in Example 5.

While structured regions formed in serum free medium containing FGF-2, the presence of 50% MEDII significantly enhanced the derivation and overall appearance of the structured regions, such that some EBs were comprised of more than 80% structured material. When crater derived sfEBs or sfEBMs were seeded onto polyornithine/laminin coated slides, significantly neuralized cell cultures were observed. Embryoid bodies seeded 3-5 days after derivation developed rosette containing cultures when cultured in essentially serum free medium containing FGF-2, with or without 50% MEDII. Embryoid bodies that had been derived in the presence of MEDII were seeded in the presence of MEDII, while embryoid bodies that had been derived without MEDII were seeded without MEDII. Without MEDII, the proportion of rosettes observed was from around 10-30% of the colony area. In the presence of MEDII, the proportion of rosettes observed increased to around 50-80% of the colony area. Other undefined cell types were usually present in both conditions, but comprised a higher proportion of the culture when MEDII was not included in the medium.

When crater derived embryoid bodies were seeded after prolonged suspension culture, more than one week after derivation, or after several passages of the structured material and more than one month culture, extensive networks of neurons were observed deriving from colonies. Immunocytochemical staining demonstrated that neural progenitors (Nestin⁺/Sox1⁺), and/or semi-differentiated neurons (Nestin⁺/Sox1⁺/radial Map2⁺), differentiated neurons (Map2⁺), and presumptive glial cells (Nestin⁺) were present in these cultures and corresponded to morphological observations of these cell types. In general, the proportion of cultures differentiating to rosettes and neurons was higher when embryoid bodies were derived from crater HESC colonies compared to multilayered HESC colonies. The presence of 50% MEDII enhanced the generation and/or proliferation of structured regions of embryoid bodies, and rosettes when seeded onto a polyornithine/laminin matrix.

Dopaminergic Differentiation

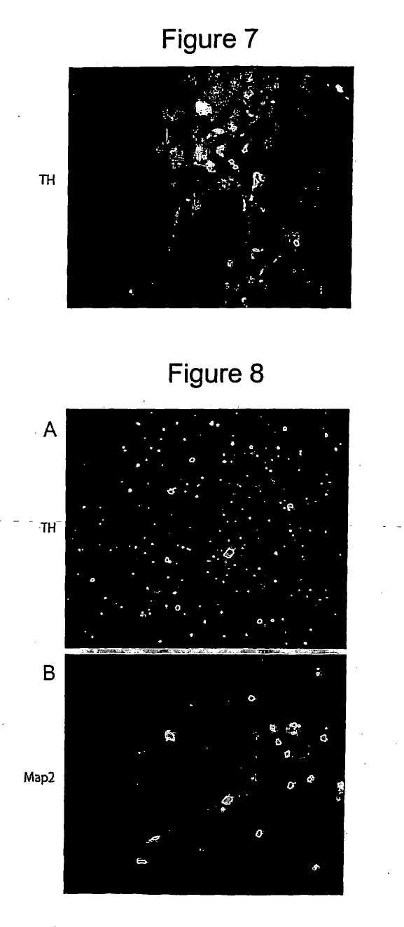

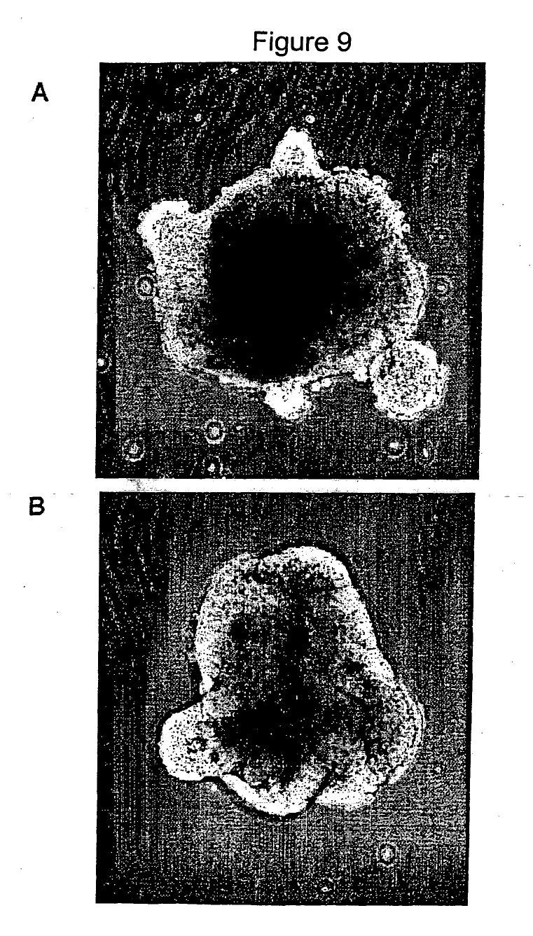

To examine the level of dopaminergic differentiation in the seeded serum free embryoid body cultures derived from crater cells, fluorescent immunocytochemical staining was performed using a sheep anti-Tyrosine Hydroxylase (TH) antibody (Pel-Freez, #P60101-0, 1:500 dilution). Isolated TH+ neurons and networks of TH+ neurons were observed in crater derived serum free EBM seeded colonies (FIG. 7) and Map2+/TH+ neurons were observed (FIG. 8). FIG. 7 shows a network of anti-Tyrosine Hydroxylase stained neurons in a crater derived sfEBM plated onto a polyornithine/laminin matrix. FIGS. 8A-8B show co-expression of Tyrosine Hydroxylase (FIG. 8A) and Map2 (FIG. 8B) shown by fluorescent immunostaining of neurons in a crater derived sfEBM plated onto a polyornithine/laminin matrix. FIGS. 9A-9B show crater derived serum free embryoid bodies at day 7 in suspension in (FIG. 9A) DMEM/F12/N2/FGF-2 and (FIG. 9B) DMEM/F12/N2/FGF-2/50% MEDII.

### Example 7

Reduction in the Level of Oct4 Protein in Differentiated HESCs

The Oct4 transcription factor is a tightly regulated marker of pluripotency in the mouse, and expression of Oct4 mRNA in human inner cell mass and ES cultures has been confirmed (Hansis et al., 2000, Mol. Hum. Reprod. 6(11), 999-1004, and Reubinoff et al., Nature Biotech. 2000, 18, 399-404). However, the restriction of Oct4 protein to pluripotent cells in humans has not been examined thoroughly. Manually passaged HESC cultures containing domed or cratered colonies were stained with anti-Oct4 antibodies.

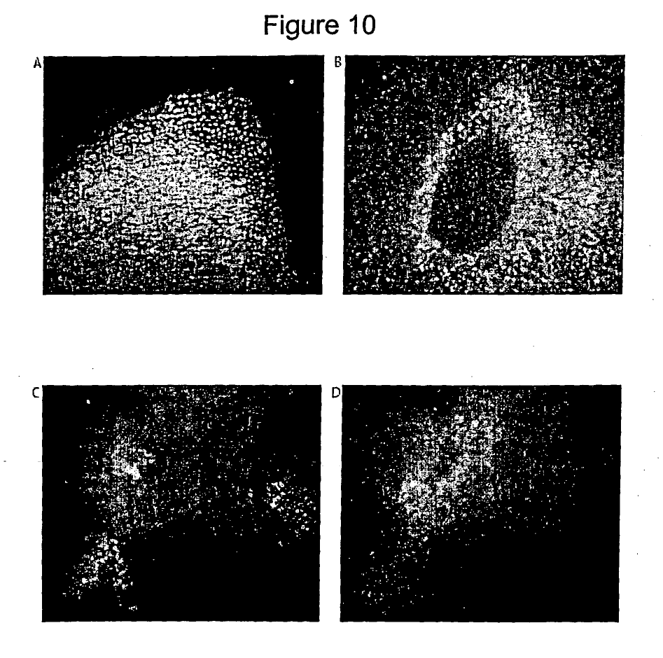

It was observed that the Oct4 protein is expressed at high levels in undifferentiated HESCs (FIG. 10A) and that levels of the Oct4 protein are down-regulated following differentiation (FIG. 10B). An unexpected characteristic of immunostaining in the culture systems analyzed was that differentiated human cells retained a reduced but detectable level of Oct4. However, when seeded sfEBM cultures were fixed and immunostained, a process that maintains the morphology of a culture, the difference between the two types of Oct4 expression was clearly distinguishable. High level Oct4 expression was only observed as bright nuclear staining in tightly packed but evenly spaced cells. Therefore immunostaining for Oct4 expression during neural differentiation in embryoid bodies was a suitable assay for the presence of residual compartments of pluripotent cells.

To monitor the persistence of pluripotent cells during sfEBM differentiation, essentially serum free embryoid bodies were generated from domed HESC colonies or monolayer crater ES cells. The sfEBMs were grown in suspension for 3-7 days, seeded onto polyornithine/laminin coated chamber slides, cultured for 3-5 days in the same medium and fixed for immunostaining. The presence of residual nests of pluripotent cells was demonstrated by clusters of high level Oct4 immunostaining amongst the generalized low level of Oct4 staining seen in the neuralized culture (FIG. 10C). The Oct4 immunoreactivity was nuclear-specific. High level Oct4 expression was not associated with the neural rosettes, which were visualized by the characteristic radial pattern of nuclei stained with DAPI (FIG. 10D). The presence of nests of residual pluripotent cells was still observed in sfEBMs that were cultured for over one month, with several passages specifically attempting to purify the neural rosette material, highlighting the persistent nature of these pluripotent cells and their implied teratoma forming potential when transplanted.

### Example 8

SSEA4 Selection and Protease Passaging Techniques Generate a Homogeneous Cell Population from ES Cells

Methods

Embryoid bodies were generated from SSEA4 selected and bulk passaged cells as described in Example 5.

Immunostaining

Immunostaining was performed as described in Example 4 for nestin and Oct4.

For immunostaining with SSEA1, SSEA3, SSEA4, Tra1-60, and Tra1-81, samples were washed in block buffer (3% goat serum, 1% PVP in PBS) for 30 minutes, and then were incubated with the appropriated dilution of the primary antibody, or combination of antibodies for 4-6 hours at room temperature. The primary antibodies used were anti-SSEA1, a mouse IgM antibody (Developmental Studies Hybridoma Bank, Catalog # MC-480), undiluted; anti-SSEA3, a rat IgM antibody (Developmental Studies Hybridoma Bank, Catalog # MC-631), undiluted; anti-SSEA4, a mouse IgG3 antibody (Developmental Studies Hybridoma Bank, Catalog # MC-813-70), undiluted; anti-Tra-1-60 (a gift from Peter Andrews), undiluted; and anti-Tra-1-81, (a gift from Peter Andrews), undiluted. The cells were then washed in wash buffer (PBS) 3 times for 5 minutes each. The remainder of the immunostaining protocol was performed as described in Example 5.

Results

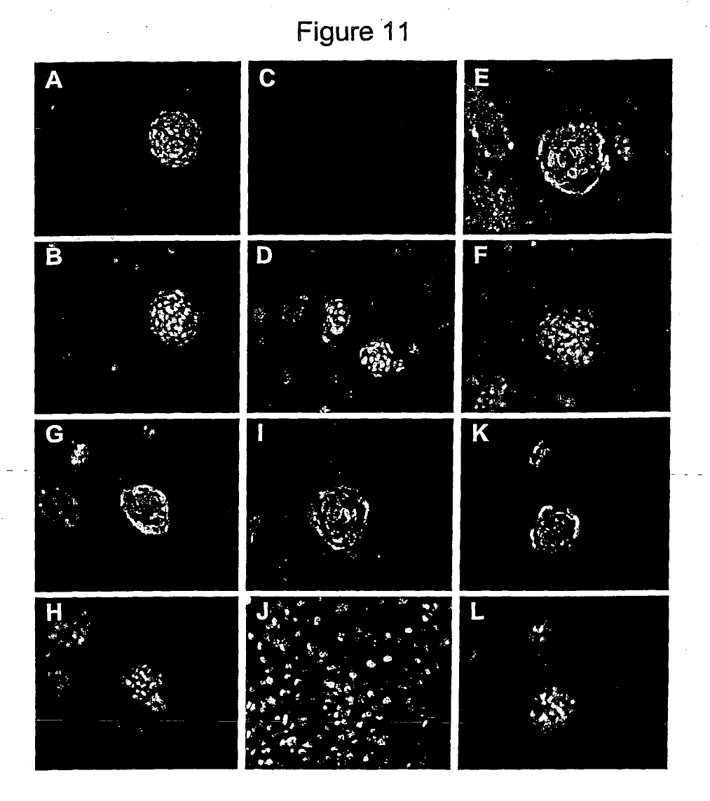

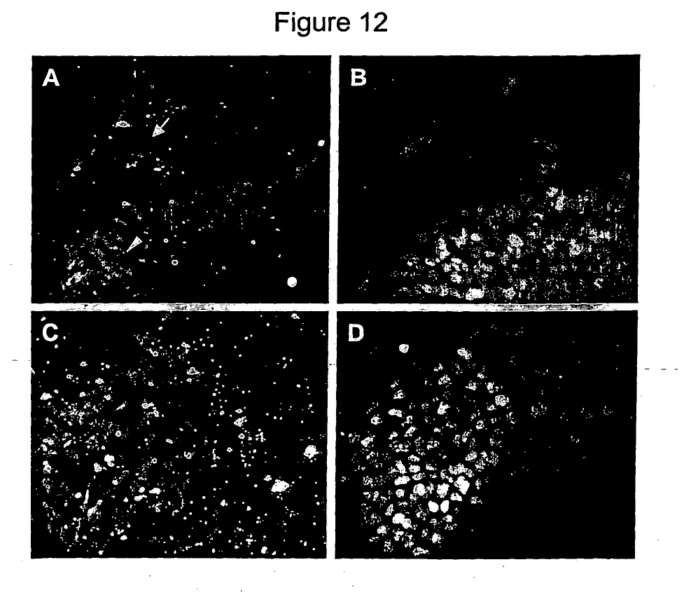

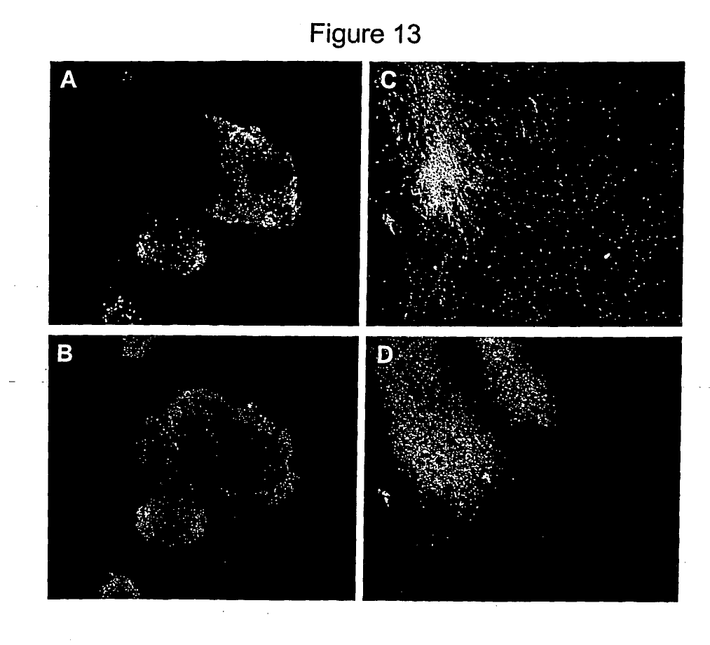

Sorted HESCs contained the expected pattern of marker expression for undifferentiated pluripotent cells: SSEA4⁺, Oct4⁺, Tra-1-60⁺, Tra-1-81⁺, SSEA3⁺, and SSEA1⁻ (FIG. 11). Unexpectedly, SSEA4 selected HESC also expressed the neural progenitor marker Nestin (FIG. 12). Manually passaged HESC cultures are typically heterogeneous, demonstrated by colonies that contained a ring of cells expressing nestin that surrounded the bulk of the colony which did not exhibit nestin expression (FIGS. 12A, and 12B). In comparison, SSEA4 selected HESCs showed uniform nestin expression (FIGS. 12C, and 12D). Nestin is a intermediate filament protein that has a distinct pattern in neural progenitor cells. Nestin staining in SSEA4 selected HESCs was organized into a uniformly distributed filamentous staining. The lack of nestin expression in the bulk of manually passaged HESCs in contrast to the uniform nestin staining in SSEA4 selected HESCs indicated that this bulk passaged population, while identical to manually passaged HESCs with regard to expression of markers of pluripotency, could be a downstream cell population with some pre-neural stem cell gene expression characteristics. However, nestin may not be a tightly restricted neural progenitor marker (see Kachinsky et al., 1994 Dev. Biol., 165(1):216-28; Wroblewski et al., 1996 Ann. NY Acad. Sci. 8(785):353-5; Wroblewski et al., 1997 Differentiation, 61(3):151-9; and Mokry and Nemecek 1998, Acta Medica, 41(2):73-80).

### Example 9

MEDII Enhanced Differentiation of SSEA4 Selected ES Cells

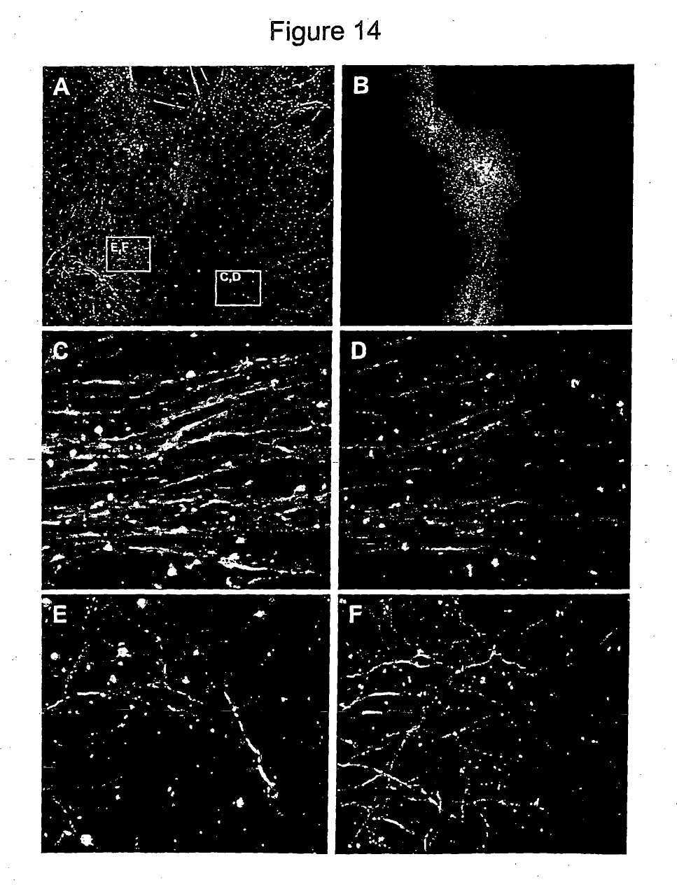

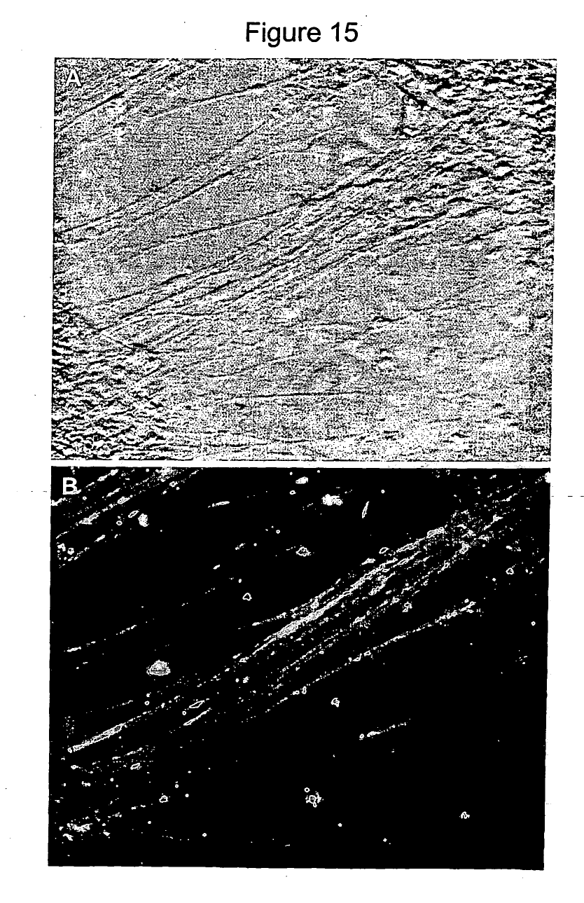

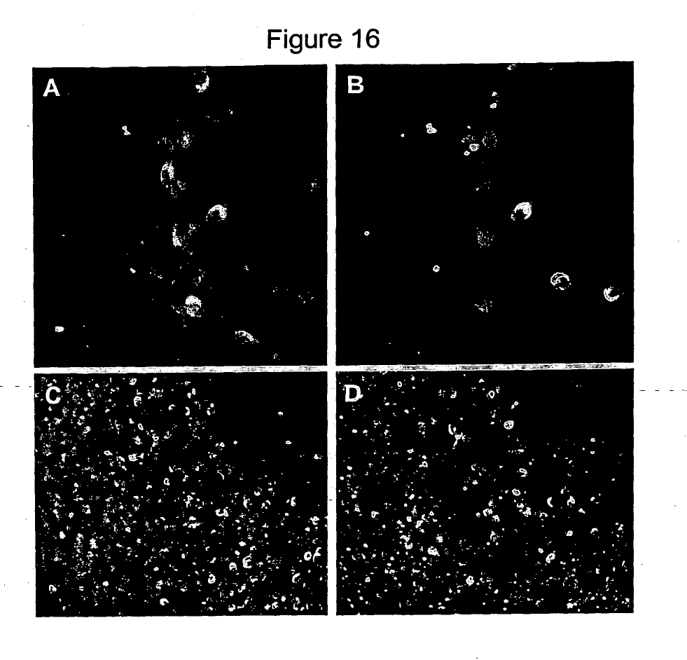

The application of 50% MEDII to embryoid bodies derived from SSEA4 selected bulk passaged cells improved the neural differentiation significantly (FIG. 13). Without MEDII, extensive TH⁺ networks were present, but the proportion of the culture that did not contain neurons and was presumably a non-neural background cell type varied between approximately 30 and 90%. In the presence of MEDII, a consistently high proportion of the culture contained TH⁺ neurons, with the background of non-neural regions that was negative for the neuronal marker βIII-Tubulin typically lower than 10%. It was not determined whether the effect of MEDII induced more efficient neuralization or inhibited the generation of non-neural cell types. Furthermore, neurons growing in the presence of MEDII exhibited much longer cellular extensions and they appeared more developed and differentiated than neurons in cultures exposed to FGF2 alone. Under this differentiation scheme, a very high proportion of all neurons, greater than 90%, expressed Tyrosine Hydroxylase (TH), the rate limiting enzyme in dopamine biosynthesis and the standard marker for dopaminergic differentiation. This proportion was determined by analysis of double staining of neural extensions for βIII-Tubulin and TH (FIG. 14), and overlaying Hoffman images with TH immunofluorescence (FIG. 15). The increase in the proportion of TH⁺ neurons in MEDII treated differentiations appeared to be due to the overall increase in neuronal differentiation, rather than an effect on the proportion of neurons that were dopaminergic, because the proportions of neurons that were TH⁺ in differentiations not exposed to MEDII was equally high. Another marker of DA cells, VMAT, was expressed in similarly high proportions of cells within the sfEBM cultures. TH⁺/VMAT⁻, TH⁻/VMAT⁺ and TH⁺/VMAT⁺ cells were observed (FIG. 16), possibly indicating temporal variability in the induction of expression of these markers prior to being co-expressed.

### Example 10

Dopamine Release Assays Using sfEBM Cultures

Methods

Dopamine released by depolarized neural cultures was detected by using a Catecholamine-Enzyme Immunoassay (Labor Diagnostika Nord), a clinical diagnostic kit for determination of Dopamine in Plasma and Urine, according to the manufacturer's instructions. The experimental sample was comprised of sfEBMs that had been derived, seeded to polyornithine/laminin coated slides at day 25 and cultured to day 30. Cells were depolarised by exposure to 300 μl 56 mM KCl in minimal MEM (Gibco) per well, for 15 minutes. The medium was removed and frozen.

The dopamine assay was performed as follows: (A) Dopamine was first extracted from the sample using a cis-diol-specific affinity gel, followed by acylation to N-acyldopamine. The supplied standards and 300 μl test sample were pipetted into wells of the cis-diol-specific affinity gel coated plate. 50 μl assay buffer containing 1 M HCl was added to the wells, followed by 50 μl extraction buffer. The plate was covered and incubated for 30 minutes at RT on an orbital shaker (600 rpm). The liquid was decanted, 1 ml wash solution added and the plate was shaken for 5 minutes at 600 rpm. The liquid was decanted and the wash repeated. 150 μl acylation buffer, then 25 μl acylation reagent was added to the wells, followed by shaking at RT for 15 minutes at 600 rpm. The liquid was decanted and 1 ml wash solution added to wells, followed by shaking for 10 minutes at RT at 600 rpm. The liquid was decanted and 150 μl 0.025 M HCl was added to wells to elute N-acyldopamine. 20 μl of the supernatant was used for the determination of dopamine. (B) The N-acyldopamine was converted enzymatically to N-acyl-3-methoxytyamine followed by a competitive Dopamine-EIA. Acylated dopamine in suspension competes with dopamine attached to the solid phase of a microtiter plate for a limited number of antiserum anti-dopamine binding sites until equilibrium is reached. Free antigen and antibody complexes are removed by washing, and antibody complexed with the solid phase dopamine is detected using a secondary antibody conjugated with peroxidase, using TMB as a substrate and detected at 450 nm. The amount of antibody bound to the solid phase is inversely proportional to the dopamine concentration of the sample.

The enzyme solution, catechol-O-methlytransferase, was made no longer than 15 minutes prior to use, and was prepared by reconstitution with 1 ml distilled water, followed by adding 0.3 ml Coenzyme, S-adenosly-L-methionine, and 0.7 ml Enzyme buffer. 25 μl of the enzyme solution was pipetted to assay wells, followed by 125 μl of 0.025 M HCl into the wells for the standards and controls. 10 μl of the extracted standards, controls, two supplied patient urine samples and 125 μl of the extracted sfEBM sample was added to the appropriate wells followed by incubation at 37° C. for 30 minutes. 50 μl anti-dopamine antiserum was added to all wells and shaken at room temperature for 2 hours at 400 rpm. The wells were aspirated and washed twice with 300 μl wash buffer per well. 100 μl secondary antibody enzyme conjugate was added to the wells and shaken for 30 minutes at room temperature at 400 rpm. The wells were aspirated and washed 3 times. 100 μl substrate was added to each well and shaken for 35 minutes at room temperature at 400 rpm in the dark. 100 μl stop solution was added to each well and the absorbance a 450 nm was read within 10 minutes. The absorbance for each standard, control and sfEBM sample were normalized for dilution and were plotted with the linear absorbance of the standards along the y-axis versus log of the standard concentrations in pg/ml along the x-axis.

Results

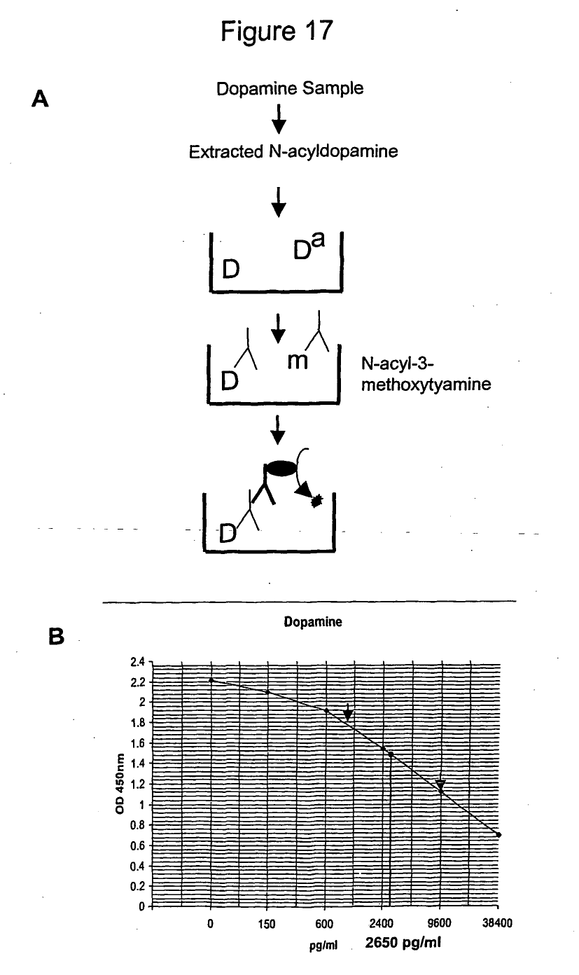

sfEBM cultures were tested for the production and release of dopamine in response to KCl, a depolarizing agent. Cultures were treated with 56 μM KCl for 15 minutes and the culture supernatant assayed for the presence of dopamine using a specific competitive ELISA. A seeded sfEBM culture supernatant contained approximately 2657 pg/ml dopamine after depolarization (FIG. 17B), indicating that dopamine was synthesized by cells within the culture and released when treated with KCl. This value does not indicate the absolute level of dopamine produced, as dopamine levels would be affected by the number of dopaminergic cells seeded as embryoid bodies, their relative level of differentiation with regard to dopamine biosynthetic pathways and vesicle production, and the volume and subsequent dilution of the KCl supernatant. However, this value was similar to the 600 pg/ml found for cultures containing mouse DA neurons (Kim et al., 2002 Nature 418: 50-56), and it also fell between two unknown control samples supplied with the kit, although these values are not directly comparable due to the above reasons.

### Example 11

Differentiation of SSEA4 Selected HESCs in the Presence of Proline

To test their neural differentiation capacity in the presence of proline, SSEA4 selected HESCs were differentiated in essentially serum free conditions as embryoid bodies.

Methods

Essentially serum free embryoid bodies were generated from bulk passaged monolayer HESC colonies as described in Example 5, in the presence of 4 ng/ml FGF2 and 100 μM Proline, or in 4 ng/ml FGF2 with MEDII conditioned medium as a positive control.

Serum free embryoid bodies were cultured in suspension for 17 days, and were cut into pieces and seeded onto polyornithine/laminin coated slides at day 10 or 17. The explants were cultured on slides for 5 days prior to fixation at day 15 or 22, for immunostaining with anti-βIII-Tubulin and anti-Tyrosine Hydroxylase antibodies.

Results

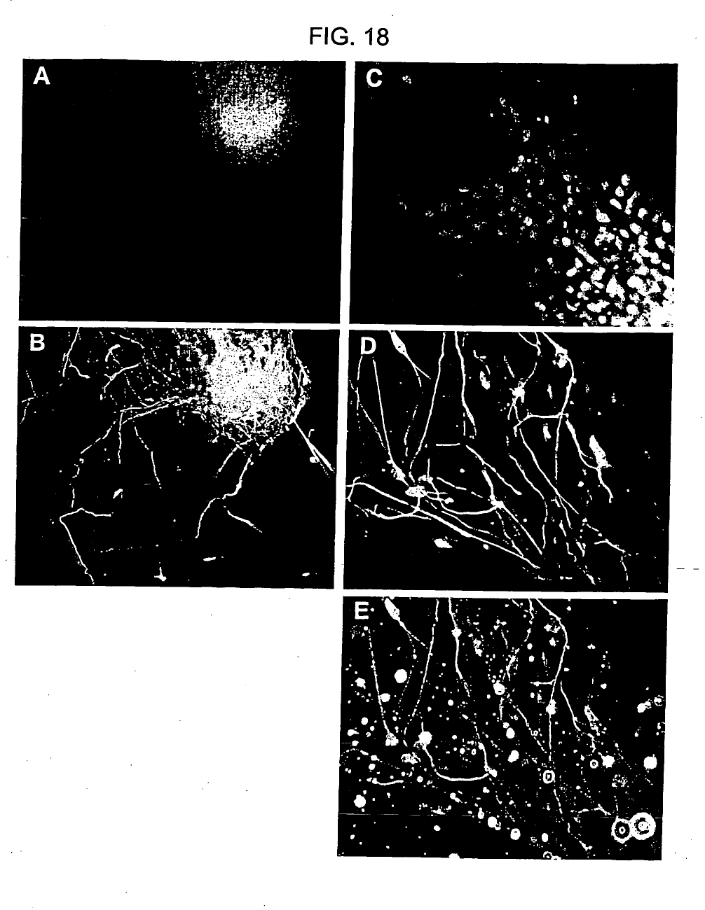

Serum free embryoid bodies grown in FGF2 and 100 μM proline (sfEBP) differentiated to neurons as observed by morphological and immunofluorescent staining of seeded pieces (FIG. 18). Dense networks of βIII-Tubulin⁺ cells were observed in the majority of seeded pieces (FIGS. 18A, and 18B). A proportion of seeded EB pieces, less than 30%, did not exhibit large networks of βIII-Tubulin⁺ cells and could represent undifferentiated neural precursors, other neural cell types, or non-neural cells. Double immunofluorescent staining indicated that greater than 90% of the neurons generated were dopaminergic, co-expressing βIII-Tubulin and TH (FIGS. 18C, D, and E). This level of dopaminergic differentiation was consistent with that observed with bulk passaged SSEA4 selected HESCs differentiated in the presence of FGF2/MEDII. Unlike sfEBMs, sfEBPs did not flatten when pieces were seeded, and generally remained in a more globular structure. As noted previously, sfEBMs exhibit large outgrowths of a monolayer cell type(s), which neurons and neural extensions grew on top of. Therefore, sfEBM cultures exhibited long neuron extensions radiating from seeded pieces, which was not as pronounced in sfEBP pieces. Therefore the effect of proline on the neural differentiation was pronounced, but did not mimic all the effects of MEDII. However, it is not clear if the proliferation of the monolayer cell type(s) will be beneficial for cell transplantations, and could effectively lower the proportions of neurons within the total culture, despite it being beneficial for in vitro differentiation of neural processes.

### Example 12

Differentiation of SSEA4 Selected HESCs in Differing Media for Formulations

To test their neural differentiation capacity in the presence of different media formulations, SSEA4 selected HESCs were differentiated in essentially serum free conditions as embryoid bodies.

Methods

Essentially serum free embryoid bodies were generated from bulk passaged monolayer HESC colonies as described in Example 5, in the following media formulations:

| | | βIII-Tubulin | TH positive |
|---|---|---|---|
| | Media Formulation | positive cells | cells |
| A | minimal medium (DMEM, | Not | Not |
| | N2, L-Glutamine, | determined | determined |
| | Penicillin, Streptomycin) | | |
| B | minimal medium with | 24% | Not |
| | 4 ng/ml FGF2 | | determined |
| C | minimal medium with | 73% | 51% |
| | 100 μM Proline | | |
| D | minimal medium with | 63% | 60% |
| | 200 μM Proline | | |
| E | minimal medium with | 31% | 58% |
| | 100 μM Proline and | | |
| | 4 ng/ml FGF2 | | |
| F | minimal medium with | 36% | 37% |
| | 200 μM Proline and | | |
| | 4 ng/ml FGF2 | | |
| G | DMEM, F12, N2, L-Glutamine, | 50% | 52% |
| | Penicillin, Streptomycin | | |
| | and 4 ng/ml FGF2 | | |
| H | DMEM, F12, N2, L-Glutamine, | 25% | 32% |
| | Penicillin, Streptomycin, | | |
| | 4 ng/ml FGF2 and 50% MEDII | | |

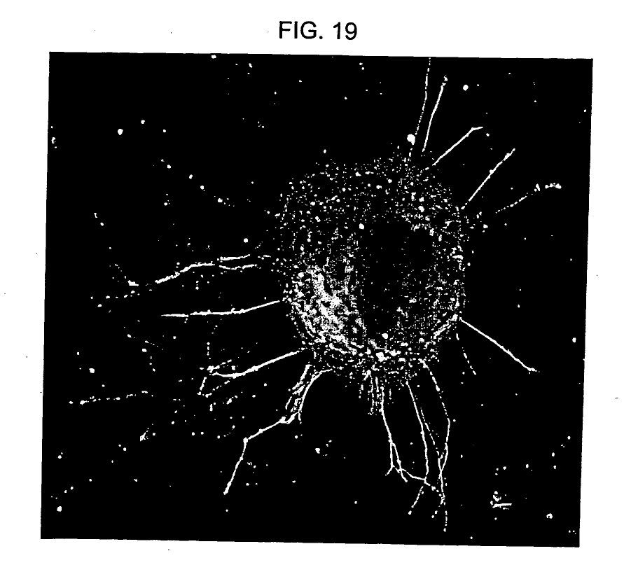

Serum free embryoid bodies were cultured in suspension for 3 weeks. Morphological differences were apparent between the cultures. Low proliferation in minimal medium (A) was observed, as well as increased cell death, with an external layer of cell death surrounding what appeared to be a viable and proliferative core of cells. Minimal medium with proline (C, D) seemed to exhibit a higher proliferation or survival rate, although still contained increased cell death compared to FGF2 containing conditions (B, E-H). Conditions B-H showed good proliferation over the course of the experiment. Serum free embryoid bodies were cultured in suspension, and were cut into pieces, seeded onto polyornithine/laminin coated slides at day 21 and fixed at day 25. Immunostaining with anti-βIII-Tubulin demonstrated the presence of extensive networks of neurons in all conditions, even in minimal medium (Condition A) that contained no FGF2, Proline, F12, or MEDII (FIG. 19). This was indicative that this differentiation protocol utilizes an intrinsic neural differentiation capacity of HESC, rather than exogenous neural inducing factors.

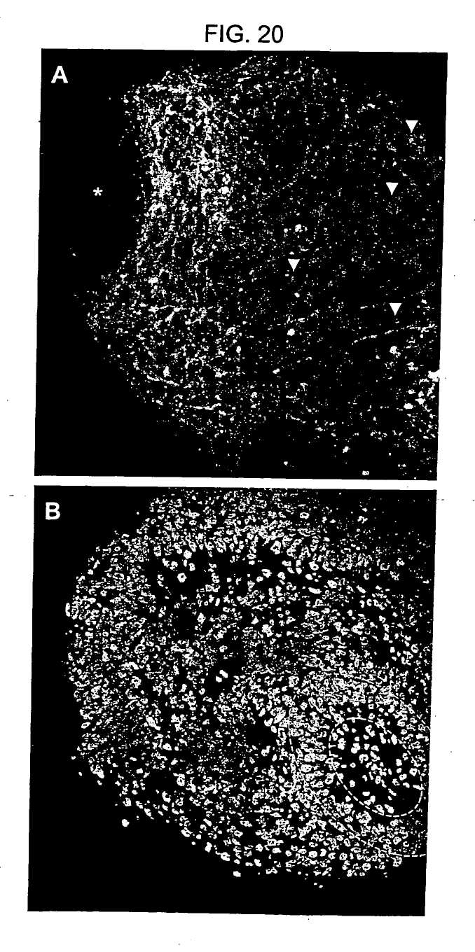

Cytospins of disaggregated serum free embryoid bodies were performed at day 21 to enable the counting of the proportion of βIII-Tubulin or TH positive cells generated in the different media formulations. βIII-Tubulin is a marker for differentiating neurons, but also known to be expressed in HESC colonies, although this expression is not neuronal-like (Carpenter et al., Exp. Neurol. 172, 383-397). Expression of βIII-Tubulin in seeded serum free embryoid bodies (FIGS. 18B, D; and FIG. 19), and in whole mount stainings of sfEBPs in suspension (FIG. 20A), was only observed in cells of overt neuronal morphology. Therefore, using this marker to count the proportion of neurons in sfEBPs is not expected to be influenced by the potential persistence of pluripotent cells. The immunostaining of these cytospins with an anti-TH antibody did not generate as strong a signal, and was therefore not likely to be as accurate as the βIII-Tubulin count.

To count proportions of neurons in serum free embryoid bodies, cytospins were immunostained with anti-βIII-Tubulin (Sigma, #T8660) or mouse anti-TH monoclonal antibodies (PelFreez Biologicals, #P80101-0), detected with alexa-488 conjugated anti-mouse secondary antibody and nuclei were stained with DAPI. Two color fluorescent images were taken under 10× magnification and merged, and double positive signals were scored as neuronal cell bodies, or TH⁺ neuronal cell bodies against the total nuclei count. A minimum of three randomly sampled fields and 250 or 100 nuclei for βIII-Tubulin or TH, respectively, were counted for each condition. The highest proportion of βIII-Tubulin positive cells was observed in L-Proline conditions (Conditions C and D), indicating the purest population of neurons generated in this comparison. The relatively lower proportion of neurons observed in FGF2/MEDII conditions (Condition H, 25%) indicated the overgrowth of the presumptive glial or glial progenitor monolayer cell type observed morphologically, rather than a reduced total number of neurons. The presence of a lower proportion of neurons in any condition containing FGF2 (Conditions B, E-H) presumably reflected the known activity of this factor in maintaining undifferentiated neural progenitors (Okabe et al., Mech Dev. 1996: 59(1):89-102).

This data indicated that neuronal differentiation occurred in suspension, and sfEBPs in particular were likely to be a mix of neural precursors and differentiating neurons. L-Proline media (Conditions C and D) appeared to exhibit the purest population of neurons, at more than 50% of the cells in a sfEBP, but it was not determined if these cells were as differentiated as observed previously in seeded sfEBM, where there are non-neuronal cell types for neurites to grow on. Where analyzed, immunostaining of cytospins with anti-TH also revealed similar proportion of TH⁺ neurons in each condition as total neurons, given the caveat of the lower confidence of the accuracy of the count. Regardless, counting of TH⁺ cell bodies indicated that the large majority of neurons in all the conditions tested were TH⁺. It is likely that this analysis will be improved as the cytospin immunostain assay for TH is optimized further. An example of this would be to develop a triple stain assay for TH/βIII-Tubulin/DAPI.

The differentiation of βIII-Tubulin positive neurons in all the conditions, including minimal chemically defined medium (Condition A), indicated that this system was based on the intrinsic capacity of HESC to differentiate to neurons, rather than the addition of exogenous “neural inducing” factors. In this scenario, the activities of L-proline, FGF2 and MEDII could be related to the proliferation and survival of cell types generated intrinsically within the system. Alternatively, components of the N2 supplement (insulin, transferrin, progesterone, selenite and putrescine) could effect a neural inducing activity. However, these components, apart for transferrin, were tested and shown to not play a significant role in neural specification in a monolayer system of mouse ES cell differentiation (Ying et al., 2003 Nat. Biotech. 21:183-186).

### Example 13

Differentiation of SSEA4 Selected HESCs in Various Concentrations of L-Proline

To test their neural differentiation capacity in the presence of a range of L-Proline concentrations, SSEA4 selected HESCs were differentiated in essentially serum free conditions as embryoid bodies.

Methods

Essentially serum free embryoid bodies were generated from bulk passaged monolayer HESC colonies as described in Example 5, in the presence of the media set out below.

| | Media Formulations |
|---|---|
| A | Minimal medium (DMEM, N2, L-Glutamine, |
| | Penicillin, Streptomycin) |
| B | Minimal medium with 5 μM Proline |
| C | Minimal medium with 50 μM Proline |
| D | Minimal medium with 100 μM Proline |
| E | Minimal medium with 500 μM Proline |

Essentially serum free embryoid bodies formed in the presence of proline containing medium are termed sfEBPs. sfEBPs were cultured in suspension for three weeks, and were passaged by manual cutting at around the 2 week mark. sfEBPs exhibited a high level of cell death throughout the first 3 weeks of suspension culture, with an outer layer of dead cells and generally slow proliferation when compared to EB formation in FGF2/MEDII conditions in previous experiments. At around 3 weeks, sfEBPs exhibiting low cell death and distinct neural rosette structures/folds were observed in all conditions. The appearance of this type of sfEBP was noticeably enhanced in the 50 μM Proline condition. A higher proportion of the sfEBPs exhibited this morphology in the 50 μM Proline condition than in other conditions, and their morphology was superior, with fewer associated dead cells and more noticeable neural rosette structures.

sfEBPs derived in 50 μM L-Proline have been passaged and maintained in a proliferative state in suspension culture for more than 7 weeks after initial derivation and 3-4 weeks after proliferation of neural rosette structures. This indicates that under these conditions there is a balance between rosette proliferation and neuronal differentiation. When seeded to polyornithine/laminin, a high proportion of DA differentiation was still exhibited. When seeded in 50 μM L-Proline, a high degree of cell death was observed in outgrowths, although good networks of βIII-Tubulin⁺ neurons were still viable. When seeded in FGF2/MEDII medium, morphologically healthy outgrowths were observed to contain neurons and cells similar to the presumed glial or glial progenitor derived from rosettes. This indicated that there were cell types within the sfEBPs that were continuously generated that could not survive in the minimal conditions. It is likely that this indicated that these cells were differentiated from rosette cells.

sfEBPs grown in 50 μM L-Proline were fixed in suspension and immuonstained with anti-βIII-Tubulin or DAPI in a wholemount assay. These sfEBPs were mounted and optically sectioned using a Leica TCS SP2 Spectral Confocal Microscope. Networks of βIII-Tubulin⁺ neurons were visualized throughout the sfEBP, as were DAPI stained neural rosettes (FIGS. 20A and B).

The high degree of cell death observed over the first 3 weeks is likely to be indicative of the continual generation of cell types that are not viable under these serum- and serum replacer-free conditions, until the generation, maturation, or adaptation of a neural rosette cell that can proliferate in minimal medium, which is enhanced in the presence of L-proline.

### Example 14

Derivation and Characterization of a Neurosphere Population from Human Embryonic Stem Cells

In this example, the method illustrated in Example 3 was essentially repeated utilising human ES cells, with the following differences. For human ES cells the MEDII conditioning was conducted using the Filtrate (<10 Kda fraction) of serum-free MEDII. In addition, human cell aggregates were formed as suspension bodies in 50% serum-free MEDII Filtrate for a period of up to 15 days with no change in media at EBM⁹. Neurospheres were then formed from embryoid bodies after disaggregation to near single cells.

Culture and Passage of Human ES Cells

SSEA4 selection of Human ES cells was carried out using magnetic bead separation and these initially sorted cells have been used in the bulk passaging protocol for these experiments as described in Example 5.

SF MEDII/Filtrate Preparation

MEDII conditioned medium was prepared as described in WO 99/53021. The filtrate fraction of MEDII was prepared by ultrafiltration through a 10⁴ Mᵣ cut-off membrane (Centricon-3 unit; Amicon) as described in WO 99/53021. Essentially the filtrate contained molecules less than 10⁴ Mᵣ.

Formation of Human Embryoid Bodies

Collagenase/trypsin passaged ES cells were prepared as a single cell suspension and seeded at a density of 150 cells/μl in low attachment TC dishes (Costar). Cell aggregates were split 1:3 at day 2 and possibly at day 3 if required. Cultures were feed daily for 9 days and on day 9 bodies were transferred to poly-L-ornithine/laminin coated 24 well trays in 0.5 ml of medium if adhesive culture was to be conducted. Another 0.5 ml media was added to each well after 24 hours incubation. Adhered cultures or suspension cultures were fed daily for a further 8 days.

Adhesive Culture for Neural Differentiation

Embryoid bodies or neurospheres/aggregates are allowed to settle onto a coated surface to allow differentiation to occur (4 to 8 days). The coating can be on a plastic surface in either a tray or a coated coverslip.

Poly-L-Ornithine/Laminin Coating

300 μl of poly-L-ornithine 0.01% solution (Sigma Cat # P4957) was added directly from bottle into each well of a 24 well tray or a 4 well tray. Trays were sealed with parafilm and incubated overnight at 4° C. Wells were rinsed 3× with sterile MQ water. Laminin (Sigma Cat# L20-20) was diluted from a 1 mg/ml frozen stock to 1 μg/ml in sterile MQ water. 300 μl of laminin (1 μg/ml) was added to each well. Trays were sealed with parafilm and incubated overnight at 4° C. Wells were rinsed 3× with sterile MQ water and then once with 1×PBS. Trays were stored with PBS at 4° C. for up to 2 to 3 weeks. Prior to seeding wells were rinsed with 1× medium by adding 1 ml of seeding media and incubating at 37° C., 5% CO₂ to equilibrate.

Preparation of Human Neurospheres

Trypsin-EGTA Disaggregation of Embryoid Bodies: a 10 ml pipette was used to transfer bodies to a yellow capped tube. Media was aspirated and 5 ml Sigma PBS added. Bodies were allowed to settle and the PBS was aspirated and 1.25 ml of EGTA (pH 7.5) was added to the tube and bodies were soaked for 5 minutes at room temperature. Solution was aspirated and 0.5 ml trypsin was added to bodies for 30 seconds. Disaggregation of the bodies was carried out by gently pipetting them up and down with a P1000 Gilson pipette until there are no large cell clumps. 0.5 ml FBS was then added and the disaggregation continued until solution was uniformly dispersed. 10 ml DMEM+5% FBS was then added and cells were spun at 300 rpm for 1 minute to remove clumps. The supernatant was transferred into a fresh yellow capped tube 15 ml conical bottom tube and cells pelleted at 1200 rpm for 4 minutes. The cell pellet was then resuspended in 100 μl of DMEM+5% FBS and a count of viable cells was performed. Dissociated cells were then seeded into a T25 flask @ 50-100 cells/μl in 6 mls of neurosphere media (NSM; DMEM/F12, B27 1:50, ITSS 1:100, Heparin (10 mg/ml) 1:1000, FGF2 ((25 mg/ml) 1:5000 dilution) and spheres allowed to form over a two-three week period. NSM was changed 50:50 every 4 days.

Passaging of Neurospheres

Disaggregation of neurospheres was conducted either using the trypsin dissociation method described above for the preparation of neurospheres or using a mechanical trituration method as follows. Using a 10 ml pipette, spheres were transferred to a 15 ml yellow capped conical bottom tube. Spheres that had attached to the flask were gently dislodged with 5 mls fresh media and added to the tube. Spheres were pelleted by centrifugation. The supernatant was removed, leaving behind approximately 200 uL and the pellet gently triturated approximately 150× using a p200 pipetteman. 5 ml of culture medium was added and centrifuged gently to remove debris. The supernatant was removed and cells were gently dissociated 10-20× to disaggregate the pellet. A viable cell count was done and cells were reseeded at 1×10³ cells/cm² (equivalent to ˜4 cells/μl).

Results

In the presence of MEDII filtrate, neurospheres were derived from EBM⁹s. If filtrate was omitted, derivation of neurospheres from EBMs was delayed until EBM¹²⁻¹⁵.

Neurospheres contained neuronal cells (NF200+ve). Neurospheres also included glial cells (GFAP⁺). TH⁺ neurons were also present after passaging.

### Example 15

Derivation and Characterization of a Neurosphere Population from Human Embryonic Stem Cells Using a Two-Stage Process

Essentially, the process using mouse ES cells, as outlined in Example 14 was repeated with some modifications using human ES cells. Human ES cell culture, cell aggregate/embryoid body formation and adherent culture was essentially as described in Example 14.

Basic Media (DMEM/F12 and ITSS or N2).

Embryoid bodies/neurospheres from human ES cells were grown without the use of MEDII conditioned media Media and supplements used were Hams DMEM/F12 (Gibco Cat # 11320-033), ITSS (Gibco Cat#17502-048) and N2 (Gibco Cat#41400-045). The media did not contain HEPES.

Comparison of Media

The ability of basic media with supplements (DMEM+N2 or ITSS) to promote neural differentiation of hES cells was compared with medium that included F12: (DMEM:F12 (1:1)+N2 or ITSS).

Initial results showed that embryoid bodies can form in either of these basic media even without FGF2. Immunohistochemistry for NF200 revealed that under both media conditions with either supplement, neurons formed. Furthermore, without the addition of a mitogen such as FGF2, there were still proliferating, Ki67 positive cells. An important distinction between the two media is that TH⁺ positive cells were present in large numbers (˜50%) in DMEM/F12 and either supplement (N2 or ITSS), but not in DMEM only with either supplement.

Trypsinised Human ES cells were seeded at approximately 10 to 20 cells/μl of media into 10 ml of neurosphere media (DMEM:F12, 10 μg/ml heparin (SIGMA), 1/50 B27 (GIBCO), 1/100 pen/strep, 1/100 ITSS) in a T75 culture flask. 10 ng/ml FGF2 was optionally added but the culture medium was preferably mitogen-free (no FGF2). Cultures were maintained in the media for 9 days after which the embryoid bodies were optionally transferred to poly-L-ornithine/laminin plates and cultured in the same media for a further 6 days. The embryoid bodies so formed (EB¹⁵), whether from adherent or suspension culture, were then triturated to near single cell form and used for either transplantation or for the formation of neurospheres/cell reaggregates.

Formation of neurospheres was achieved as described in Example 14. The neurospheres formed were seeded onto poly-L-ornithine/laminin-coated plates and allowed to adhere and differentiate. Optionally in this culture stage, neurospheres can be maintained in media containing combinations of RA, 50% MEDII or filtrate, and L-proline. In an alternative treatment, during stage A, the embryoid bodies (EB⁹) can be seeded onto poly-L-ornithine/laminin-coated culture plates and cultured for 6 to 8 days to permit neuronal differentiation.

In an alternative treatment (Stage B), neurosphere formation was achieved when embryoid bodies formed from stage A were triturated and resuspended in a minimal media (DMEM/F12 and N2 or ITSS). Optionally, this media can also include combinations of FGF, MEDII, RA and L-proline. The aggregates formed can also be seeded onto poly-L-ornithine/laminin coated plates and allowed to adhere and differentiate.

Results

In the presence of F12 media, embryoid bodies formed that when adhered and differentiated formed high numbers of TH⁺ cells. If F12 was omitted, very few TH⁺ cells were observed, however, many NF200 positive cells were present, suggesting that the absence of TH⁺ neurons was not due to an inability of neuronal cells to differentiate under these growth conditions.

The presence of FGF2 appears to have had little impact on the generation of TH⁺ cells. However, there was a more extensive outgrowth of cells around the seeded body when FGF2 was present in the media.

The neurospheres contained NF200⁺ neuronal cells, GFAP⁺ glial cells and oligodendrocytes.

### Example 16

Derivation and Characterization of a Neurosphere Population from Human Embryonic Stem Cells Using a Two-Stage Process and Minimal Media

Example 15 was repeated utilising human ES cells and a minimal media consisting of DMEM and 100 μM L-proline. The results were similar to those described in Example 5.

Human ES cell culture, embryoid body/cell aggregate formation, adherent culture and passaging to form neurospheres or cell reaggregates were essentially conducted as outlined in Example 14.

L-Proline

EB¹⁷ bodies formed in medium that contained DMEM and 100 μM L-proline were comprised of proliferating Ki67⁺ cells, NF200⁺ neuronal cells, and a high proportion (˜50%) TH⁺ cells. When the medium excluded L-proline, the TH⁺ cell content of EB¹⁷ bodies was reduced significantly. Generation of EBs with high proportions of TH⁺ cells occurred in the absence of FGF2. Cells grown in the DMEM and N2 or ITSS did not produce a significant population of TH⁺ cells.

Formation of neurospheres was achieved as described in Example 14. The neurospheres formed were seeded onto poly-L-ornithine/laminin coated plates and allowed to adhere and differentiate. Optionally in this culture stage, neurospheres are maintained in media containing combinations of RA, 50% MEDII and L-Proline. In an alternative treatment, during stage A, the embryoid bodies (EB⁹) are seeded onto poly-L-ornithine/laminin coated culture plates and cultured for 6 to 8 days to permit neuronal differentiation. The neurospheres were positive for the neurofilament marker NF200, and included glial cells that were GFAP⁺, as well as oligodendrocytes.

In an alternative treatment, embryoid bodies cultured from stage A are triturated and resuspended in a minimal media (DMEM/F12 and N2 or ITSS). Optionally this media also includes combinations of FGF, MEDII, RA and L-Proline. The aggregates formed are seeded onto poly-L-Ornithine/laminin coated plates and allowed to adhere and differentiate.

### Example 17

Study of Cell Implants in Sprague-Dawley Rats

Example 14 was repeated with certain modifications. Single Human ES cells (trypsinised) were grown in a standard suspension culture containing 50% MEDII filtrate in the presence of FGF2. At day 9 the embryoid bodies formed (EBM⁹) were transferred to poly-n-ornithine/laminin coated plates in the same serum-free MEDII filtrate culture medium, maintained for a further 8 days and allowed to adhere. The use of FGF2 in the serum-free MEDII filtrate culture medium was optional. The embryoid bodies so formed (EBM¹⁷) were then trypsinised to near single cell form. A cell suspension of 100,000 cells/μl was stereotaxically injected (100,000 cells/μl per animal) into the 6-OHDA lesioned striatum of eight Sprague-Dawley rats. A group of 5 Rats was also included that did not receive cell implants, and acted as sham controls. Rats were maintained under conditions of immunosuppression using Cyclosporin A (10 mg/kg) for a period of 8 weeks and rotational data was collected. Grafted human cells were detected using a human Alu-repeat DNA detection system.

After the 8 week period, the 8 implanted rats showed a statistically significant reduction in their rotational scores compared to the control group, with Single Factor ANOVA, p=0.047.

Immunohistochemical characterization of the human cell implants revealed neural lineages such as glial cells, and low numbers of neural cells positive for the dopaminergic neurone marker, Tyrosine Hydroxylase (TH⁺). Implanted human cells from one rat (N274) expressed the neuronal marker GFAP an astrocyte/glial lineage marker, DAPI, a non-specific nuclear marker and an Alu DNA probe in situ specific for detection of human cells, thereby showing that implanted human cells were able to differentiate to glia. Other implanted human cells from another rat (N278) expressed the neural precursor marker Nestin, human specific Alu DNA probe in situ, and a general nuclear marker DAPI. Immunohistochemistry was preformed on the implants to detect the presence of TH⁺ cells using chromogens. At least one rat (N278) contained implanted cell that expressed the dopaminergic neurone lineage marker Tyrosine Hydroxylase. A small cluster of TH⁺ cells were detectable with clearly staining cell bodies.

### Example 18

Study of Cell Implants in Sprague-Dawley Rats

Example 17 was repeated utilising minimal culture media (DMEM:F12, and ITSS or N2) with or without 10 μg/ml FGF2 in both stages A and B. This produced embryoid bodies at days 15 to 17 (EB 15 to 17) containing high numbers of TH positive neuronal cells (see Example 15).

The cells were trypsinised to an essentially single cell suspension. 1 μl containing approximately 100,000 cells, was transplanted into a rat model as described above. FGF2 was not included in the culture medium used to prepare cells for transplant, however, inclusion of FGF2 in the culture medium is optional. In one embodiment, EB⁹s are cultured on laminin/polylornithine coated plates for a further period of up to 8 days.

### Example 19

Identification of Neural Cells with Specific Markers

Neural cells produced according to the essentially serum free media methods as described above are identified by expression of detectable markers. The markers used in this example include tyrosine hydroxylase (TH), which is the rate limiting enzyme in dopamine biosynthesis. Another marker is the aromatic amino acid decarboxylase (AADC, also known as dopa decarboxylase) which is the second enzyme in the pathway for dopamine synthesis. Also detected is vesicular monoamine transporter (VMAT), the vesicular transporter that packages dopamine (and other catecholamines) into synaptic vesicles. Therefore, VMAT is required for dopamine release. Further detected is dopamine transporter (DAT), the plasma membrane transporter that brings dopamine back into the cell after it has been released. DAT is likely the most specific marker for dopaminergic neurons. The co-expression of these markers within a neuron is required for the synthesis, vesicular packaging and reuptake of dopamine, consistent with the normal function of a dopaminergic neuron.

For immunostaining, the neural cells are rinsed with 1×PBS and fixed in 4% paraformaldehyde, 4% sucrose in 1×PBS for 30 minutes at 4° C. The cells are then washed in 1×PBS and stored at 4° C. To perform immunostaining, the cells are washed in block buffer (3% goat serum, 1% polyvinyl Pyrolidone, 0.3% Triton X-100 in wash buffer) for 30 minutes, and then incubated with the appropriate dilution of the primary antibody, or combination of antibodies for 4-6 hours at room temperature. The primary antibodies are anti-TH, a sheep monoclonal antibody (Pel-Freez, Catalog # P60101-0) at 1:500 dilution; anti-VMAT2, a rabbit monoclonal antibody (Chemicon, Catalog # AB1767) at a 1/500 dilution; anti-DAT, a rabbit monoclonal antibody recognizing extracellular loop 2 of DAT (Chemicon, Catalog # AB5802) at a 1/100 dilution; and anti-DAT, a rat monoclonal antibody recognizing an intracellular DAT epitope (Chemicon, Catalog # MAB369) at a 1/200 dilution. For anti-AADC, for a primary antibody, one of a rabbit anti-dopa decarboxylase polyclonal antibody (Chemicon AB136 or Chemicon AB1569), and a sheep anti-dopa decarboxylase polyclonal antibody (Chemicon AB119) is used.

The cells are then washed in wash buffer (50 mM Tris-HCL pH 7.5, and 2.5 mM NaCl; 3 times for 5 minutes each). The cells are then incubated for a minimum of 2 hours in secondary antibodies diluted 1:1000, followed by washing in wash buffer. The secondary antibodies are Alexa-350 (blue), -488 (green) or -594 (red) conjugated goat anti-sheep, anti-rabbit, or anti-rat antibodies, all available from Molecular Probes. The cells can be stained with 5 ng/ml DAPI to detect cell nuclei. The cells are then washed from overnight to 2 days in a large volume of wash buffer. The slides are mounted with mounting medium and a cover slip. Slides are visualized using a NIKON TE 2000-S inverted microscope or a NIKON E1000 upright microscope with a Q Imaging digital camera.

Neural cells produced from HESCs under serum free conditions as described herein are found to be TH⁺, VMAT2⁺, DAT⁺, and AADC⁺ by immunostaining. This example demonstrates the successful in vivo production of a neural cell population from pluripotent HESC that is believed to be capable of dopaminergic production. Therefore, such a neural cell culture is expected to be a viable candidate for therapeutic transplantation to alleviate conditions characterized by dopaminergic deficiency, such as Parkinson's disease.

### Example 20

Identification of Neural Cells with Specific Markers

Neural cells produced according to the essentially serum free media methods as described above are identified by expression of detectable markers. The markers used in this example include nestin and vimentin. Preferably, the neural cells produced using the methods identified herein have the capacity to differentiate into cells of the neural lineage, including into neurons and glial cells. The neural cells types produced may include cells of the central or peripheral nervous system including but not limited to neurons, astrocytes, oligodendrocytes and Schwann cells. Neuron cell types produced in these cultures may express one or more neurotransmitter phentotypes. These include GABAergic neurons that express glutamate decarboxylase (GAD) or vesicular inhibitory amino acid transporter/vesicular gaba transporter (Viaat/Vgat); cholinergic neurons that express choline acetyltransferase (ChAT/CAT) or vesicular acetylcholine transporter (VAChT); glutamatergic neurons that express the vesicular glutamate transporter; glycinergic neurons that express the vesicular inhibitory amino acid transporter (Viaat/Vgat), noradrenergic neurons that express the norepinephrine transporter (NET); adrenergic neurons that express phenylmethanolamine N-methyl transferase (PNMT); serotonergic neurons that express tryptophan hydroxylase (TrH) or serotonin transporter (SERT); or histaminergic neurons that express histidine decarboxylase (HDC).

For immunostaining, the neural cells are rinsed with 1×PBS and fixed in 4% paraformaldehyde, 4% sucrose in 1×PBS for 30 minutes at 4° C. The cells are then washed in 1×PBS and stored at 4° C. To perform immunostaining, the cells are washed in block buffer (3% goat serum, 1% polyvinyl Pyrolidone, 0.3% Triton X-100 in wash buffer) for 30 minutes, and then incubated with the appropriate dilution of the primary antibody, or combination of antibodies for 4-6 hours at room temperature.

The cells are then washed in wash buffer (50 mM Tris-HCL pH 7.5, and 2.5 mM NaCl; 3 times for 5 minutes each). The cells are then incubated for a minimum of 2 hours in secondary antibodies diluted 1:1000, followed by washing in wash buffer. The secondary antibodies are Alexa-350 (blue), -488 (green) or -594 (red) conjugated goat anti-sheep, anti-rabbit, or anti-rat antibodies, all available from Molecular Probes. The cells can be stained with 5 ng/ml DAPI to detect cell nuclei. The cells are then washed from overnight to 2 days in a large volume of wash buffer. The slides are mounted with mounting medium and a cover slip. Slides are visualized using a NIKON TE 2000-S inverted microscope or a NIKON E1000 upright microscope with a Q Imaging digital camera.

This example demonstrates the successful in vivo production of a neural cell population from pluripotent HESCs that is believed to be capable of neurotransmitter production. Therefore, such a neural cell culture is expected to be a viable candidate for therapeutic transplantation to alleviate conditions characterized by neurotransmitter deficiency.

1. A method of producing a human neural cell comprising,
a) providing a pluripotent human cell;
b) forming an embryoid body by contacting the pluripotent human cell with an essentially serum free medium; and
c) culturing the embryoid body in an essentially serum free cell differentiation environment
to thereby produce the human neural cell.

2. The method of claim 1, wherein the essentially serum free medium of step b) comprises Ham's F12 medium.

3. The method of claim 1, wherein the essentially serum free medium of step b) comprises a MEDII conditioned medium.

4. The method of claim 1, wherein the essentially serum free cell differentiation environment of step c) comprises a MEDII conditioned medium.

5. The method of claim 1, comprising the additional steps performed after step b) and before step c):
a) dispersing the embryoid body to an essentially single cell suspension;
b) culturing the essentially single cell suspension in a suspension culture; and
c) forming a second embryoid body by culturing the essentially single cell suspension with a second essentially serum free medium, wherein the second essentially serum free medium comprises a MEDII conditioned medium.

6. The method of claim 5, wherein one or more of the essentially serum free media is essentially LIF free.

7. The method of claim 5, wherein the second essentially serum free medium comprises DMEM/F12, FGF-2 and a MEDII conditioned medium.

8. The method of claim 5, wherein the second essentially serum free medium comprises between approximately 10% to approximately 50% MEDII conditioned medium.

9. The method of claim 5, wherein the essentially serum free medium, the second essentially serum free medium, and/or the essentially serum free cell differentiation environment comprises less than 5% serum.

10. The method of claim 5, wherein the cell differentiation environment is selected from the group consisting of adherent culture and suspension culture.

11. The method of claim 1, wherein the human cell is a pluripotent human cell.

12. The method of claim 11, wherein the pluripotent human cell is selected from the group consisting of a human embryonic stem cell, a human inner cell mass (ICM)/epiblast cell, a human primitive ectoderm cell, a human primordial germ cell, a human teratocarcinoma cell.

13. (canceled)

14. (canceled)

15. The method of claim 12, wherein the human primitive ectoderm cell is an early primitive ectoderm (EPL) cell.

16. (canceled)

17. (canceled)

18. The method of claim 11, wherein the pluripotent human cell is a human embryonic stem cell, and wherein the human embryonic stem cell is passaged by selection with a SSEA4 antibody and/or with a sequential collagenase and trypsin treatment prior to forming an embryoid body.

19. The method of claim 1, wherein the human cell is a multipotent human cell.

20. The method of claim 5, wherein the MEDII conditioned medium is a Hep G2 conditioned medium.

21. (canceled)

22. (canceled)

23. (canceled)

24. The method of claim 5, wherein the MEDII medium comprises a low molecular weight component selected from the group consisting of a proline residue, and a polypeptide comprising one or more proline residues.

25. (canceled)

26. A neural cell produced by the methods of claim 1.

27. A method for treating a patient, comprising a step of administering to the patient having a neural disease a therapeutically effective amount of the neural cell of claim 26.

28. The method of claim 27, wherein the neural disease is Parkinson's disease.

29. A method of producing a partially differentiated pluripotent cell comprising culturing a pluripotent cell culture on a layer of fresh feeder cells, wherein the fresh feeder cells have been plated for less than approximately 48 hours, thereby inducing formation of a more differentiated pluripotent cell.

30. The method of claim 29, wherein the fresh feeder cells have been plated for less than approximately 24 hours.

31. The method of claim 29, wherein the fresh feeder cells have been plated for less than approximately 12 hours.

32. The method of claim 29, wherein the fresh feeder cells have been plated for less than approximately 6 hours.

33. The method of claim 29, wherein the pluripotent cell culture forms a colony after it is cultured on the layer of fresh feeder cells, and the more differentiated pluripotent cell is selected from the central region of the colony.

34. The method of claim 33, wherein the more differentiated pluripotent cell expresses less Oct4 than an embryonic stem cell.

35. The partially differentiated cell generated using the method of claim 29.

36. A neural cell culture composition comprising a population of neural cells derived in vitro from pluripotent cells, wherein the neural cells express one or more detectable markers for tyrosine hydroxylase (TH), vesicular monamine transporter 2 (VMAT2), aromatic amino acid decarboxylase (AADC) and dopamine transporter (DAT).

37. (canceled)

38. The composition of claim 36, wherein at least one of the cultured cells expresses all of the detectable markers TH, VMAT2, AADC and DAT.

39. The composition of claim 36, wherein the neural cell is a human cell.

40. A neural cell culture composition comprising a population of neural cells derived in vitro from pluripotent cells, wherein the neural cells express one or more detectable markers for nestin or vimentin, and the neural cells have the capacity to differentiate into cells of a neural lineage.

41. (canceled)

42. The neural cell culture composition of claim 40, wherein the neural lineage is selected from the group consisting of neurons, astrocytes, oligodendrocytes and Schwann cells.

43. (canceled)

44. The neural cell culture composition of claim 40, wherein the cells of the neural lineage express a neurotransmitter phenotype selected from the group consisting of a GABAergic neuron, a cholinergic neuron, a glutamatergic neuron, a glycinergic neuron, a noradrenergic neuron, an adrenergic neuron, a sertonergic neuron, and a histaminergic neuron.

45. The neural cell culture composition of claim 44, wherein the neurotransmitter phenotype is a GABAergic neuron that expresses glutamate decarboxylase and/or expresses vesicular inhibitory amino acid transporter/vesicular gaba transporter.

46. The neural cell culture composition of claim 44, wherein the neurotransmitter phenotype is a cholinergic neuron that expresses choline acetyltransferase and/or vesicular acetylcholine transporter.

47. The neural cell culture composition of claim 44, wherein the neurotransmitter phenotype is a glutamatergic neuron that expresses vesicular glutamate transporter.

48. The neural cell culture composition of claim 44, wherein the neurotransmitter phenotype is a glycinergic neuron that expresses vesicular inhibitory amino acid transporter.

49. The neural cell culture composition of claim 44, wherein the neurotransmitter phenotype is a noradrenergic neuron that expresses norepinephrine transporter.

50. The neural cell culture composition of claim 44, wherein the neurotransmitter phenotype is a adrenergic neuron that expresses phenylmethanolamine N-methyl transferase.

51. The neural cell culture composition of claim 44, wherein the neurotransmitter phenotype is a serotonergic neuron that expresses tryptophan hydroxylase or serotonin transporter.

52. The neural cell culture composition of claim 44, wherein the neurotransmitter phenotype is a histaminergic neuron that expresses histidine decarboxylase.

53. The composition of claim 40, wherein the neural cell is a human cell.


SEQUENCE LISTING (5 sequences)

SEQ ID NO: 1 - protein, 4 aa, Artificial Sequence
  Description of Artificial Sequence Synthetic peptide
GPRP

SEQ ID NO: 2 - protein, 4 aa, Artificial Sequence
  Description of Artificial Sequence Synthetic peptide
GPGG

SEQ ID NO: 3 - protein, 4 aa, Artificial Sequence
  Description of Artificial Sequence Synthetic peptide
VAPG

SEQ ID NO: 4 - protein, 4 aa, Artificial Sequence
  Description of Artificial Sequence Synthetic peptide
RPKP

SEQ ID NO: 5 - protein, 11 aa, Artificial Sequence
  Description of Artificial Sequence Synthetic peptide
RPKPQQFFGLM